(19) United States
(12) Patent Application Publication
(10) Pub. No.: US 2008/0287516 A1
(43) Pub. Date: Nov. 20, 2008

(54) Phenyl-Substituted Pyrrolidones

(75) Inventors: Baogen Wu, San Diego, CA (US); Truc Ngoc Nguyen, San Diego, CA (US); David Archer Ellis, San Diego, CA (US); Xiaohui He, San Diego, CA (US); Beth Marie Anaclerio, San Diego, CA (US); Kunyong Yang, San Diego, CA (US); Ha-soon Choi, San Diego, CA (US); Zhicheng Wang, San Diego, CA (US); Thomas H. Marsilje, San Diego, CA (US); Yun He, Shanghai (CN)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, HAMILTON (BM)

(21) Appl. No.: 11/814,480

(22) PCT Filed: Jan. 30, 2006
(86) PCT No.: PCT/US06/03217

§ 371 Date: Mar. 19, 2008

Related U.S. Application Data
(60) Provisional application No. 60/648,027, filed on Jan. 28, 2005.

(51) Int. Cl. A61K 31/416; C07D 207/12; C07D 231/56; A61P 31/18; A61K 31/4015

(52) U.S. Cl. 514/405; 548/543; 548/362.1; 548/361.5; 514/424; 514/403

(57) ABSTRACT

The present invention relates to phenyl-substituted pyrrolidones and compounds related to phenyl-substituted pyrrolidones. One use of these compounds is for the inhibition of viruses, e.g., HIV. The invention further relates to methods of malting these compounds, methods of identifying the efficacy of these compounds, and methods of using these compounds to inhibit or prevent HIV infection and related disease states such as AIDS.

### CROSS-REFERENCES TO RELATED APPLICATIONS

The present application claims priority to U.S. Provisional Patent Application No. 60/648,027, filed on Jan. 28, 2005, which is incorporated herein by reference in its entirety for all purposes.

### FIELD OF THE INVENTION

The present invention relates to phenyl-substituted pyrrolidones and compounds related to phenyl-substituted pyrrolidones. One use of these compounds is for the inhibition of viruses, e.g., HIV. The invention further relates to methods of making these compounds, methods of identifying the efficacy of these compounds, and methods of using these compounds to inhibit or prevent HIV infection and related disease states such as AIDS.

### BACKGROUND OF THE INVENTION

The Human Immunodeficiency Virus (HIV) infects millions of people globally. Cases are reported from nearly every country amounting to 40 million adults and children living with HIV/AIDS worldwide. In 2001, 5 million people were newly infected with HIV, and there were 3 million adult and child deaths due to HIV/AIDS. A full third of those people living with AIDS are aged 15-24. (World Health Organization, 2001). HIV/AIDS treatments exist, however, the drugs currently used in treatment modalities exhibit numerous side effects, require prolonged treatment that often induces drug resistance, and do not result in complete eradication of the virus from the body.

The disease AIDS is the end result of an HIV-1 or HIV-2 virus following its own complex life cycle. The virion life cycle begins with the virion attaching itself to the host human T-4 lymphocyte immune cell through the bonding of a glycoprotein on the surface of the virion's protective coat with the CD4 glycoprotein on the lymphocyte cell. Once attached, the virion sheds its glycoprotein coat, penetrates into the membrane of the host cell, and uncoats its RNA. The virion enzyme, reverse transcriptase, directs the process of transcribing the RNA into single-stranded DNA. The viral RNA is degraded and a second DNA strand is created. The now double-stranded DNA is integrated into the human cell's genes and those genes are used for virus reproduction.

At this point, RNA polymerase transcribes the integrated DNA into viral RNA. The viral RNA is translated into the precursor gag-pol fusion polyprotein. The polyprotein is then cleaved by the HIV protease enzyme to yield the mature viral proteins. Thus, HIV protease is responsible for regulating a cascade of cleavage events that lead to the virus particle's maturing into a virus that is capable of full infectivity.

The typical human immune system response, killing the invading virion, is taxed because the virus infects and kills the immune system's T cells. In addition, viral reverse transcriptase, the enzyme used in making a new virion particle, is not very specific, and causes transcription mistakes that result in continually changed glycoproteins on the surface of the viral protective coat. This lack of specificity decreases the immune system's effectiveness because antibodies specifically produced against one glycoprotein may be useless against another, hence reducing the number of antibodies available to fight the virus. The virus continues to reproduce while the immune response system continues to weaken. Eventually, the HIV largely holds free reign over the body's immune system, allowing opportunistic infections to set in and without the administration of antiviral agents, immunomodulators, or both, death may result.

There are at least three critical points in the virus's life cycle which have been identified as possible targets for antiviral drugs: (1) the initial attachment of the virion to the T-4 lymphocyte or macrophage site, (2) the transcription of viral RNA to viral DNA (reverse transcriptase, RT), and (3) the processing of gag-pol protein by HIV protease.

Inhibition of the virus at the second critical point, the viral RNA to viral DNA transcription process, has provided a number of the current therapies used in treading AIDS. This transcription must occur for the virion to reproduce because the virion's genes are encoded in RNA and the host cell reads only DNA. By introducing drugs that block the reverse transcriptase from completing the formation of viral DNA, HIV-1 replication can be stopped.

A number of compounds that interfere with viral replication have been developed to treat AIDS. For example, nucleoside analogs, such as 3′-azido-3′-deoxythymidine (AZT), 2′,3′-dideoxycytidine (ddC), 2′,3′-dideoxythymidinene (d4T), 2′,3′-dideoxyinosine (ddI), and 2′,3′-dideoxy-3′-thia-cytidine (3TC) have been shown to be relatively effective in halting HIV replication at the reverse transcriptase (RT) stage.

The use of pyrrolidones to treat respiratory disorders is known in the art. See, for example, Keller et al., Chem. Pharm. Bull. 49(8): 1009-1017 (2001); and Bacher et al., Bioinorg. Med. Chem. Lett. 8: 3229-3234 (1998). The use of the disclosed pyrrolidones to treat HIV and related disorders is not suggested by either of these references.

Even with the current success of reverse transcriptase inhibitors, it has been found that HIV patients can become resistant to a single inhibitor. Thus, it is desirable to develop additional inhibitors to further combat HIV infection.

### SUMMARY OF THE INVENTION

It has now been discovered that pyrrolidones having novel structures are effective agents against HIV. Selected pyrrolidones of the invention are potent reverse transcriptase inhibitors. Accordingly, the present invention provides pharmaceutical formulations, and prophylactic and therapeutic treatments, diagnostic and prognostic methods and kits, and pharmaceutical screening methods that take advantage of the anti-HIV activity of the pyrrolidones.

Because the pyrrolidones of the invention inhibit HIV replication, the administration of the pyrrolidones to persons either prophylactically or therapeutically is a treatment for HIV infection. Prophylactic treatments are especially useful for persons at high risk of HIV infection. This invention provides methods of inhibiting HIV replication in a person by administering to the person a pharmaceutically effective amount of a pyrrolidone. This invention also provides pharmaceutical formulations comprising one or more pyrrolidones in a pharmaceutically acceptable carrier.

Methods of inhibiting HIV replication described above can be applied to cells being cultured in vitro, as well.

In another aspect, the present invention provides a composition comprising at least one pyrrolidone and a second therapeutic agent or agents. In one embodiment, the second therapeutic agent is used to prevent or treat HIV infection. In another embodiment, the second therapeutic is used to treat an opportunistic infection associated with HIV infection. In another embodiment, the second therapeutic agent is a protease inhibitor, a non-nucleoside reverse transcriptase inhibitor, a nucleoside reverse transcriptase inhibitor, an antiretroviral nucleoside, an entry inhibitor, or any other anti-viral agent effective to inhibit or treat HIV infection.

In another aspect, the present invention provides methods of treating or preventing HIV infection in a human comprising administering a pyrrolidone of the invention to the human in combination with a second therapeutic agent(s). In one embodiment, the second therapeutic agent is used to prevent or treat HIV infection. In another embodiment, the second therapeutic is used to treat an opportunistic infection associated with HIV infection. In another embodiment, the second therapeutic agent is a protease inhibitor, a non-nucleoside reverse transcriptase inhibitor, a nucleoside reverse transcriptase inhibitor, an antiretroviral nucleoside, an entry inhibitor, or any other anti-viral agent effective to inhibit or treat HIV infection. In another embodiment, the second therapeutic agent is selected from the group consisting of zidovudine, didanosine, stavudine, interferon, lamivudine, adefovir, nevirapine, delaviridine, loviride, saquinavir, indinavir, and AZT. In another embodiment, the second therapeutic agent is an antibiotic or acyclovir.

In another aspect, the present invention provides methods of inhibiting HIV infection in a CD4⁺ culture comprising the step of contacting the cell with a pyrrolidone of the invention, either alone or in combination with a second therapeutic agent or a combination of other therapeutic agents. In one embodiment, the therapeutic agent or agents are used to treat or prevent HIV infection. In a second embodiment, the therapeutic agent is selected from the group consisting of a protease inhibitor, a non-nucleoside reverse transcriptase inhibitor, a nucleoside reverse transcriptase inhibitor, an antiretroviral nucleoside, an entry inhibitor, and any other anti-viral agent effective to inhibit or treat HIV infection. In a third embodiment, a therapeutic agent is selected from the group consisting of zidovudine, didanosine, stavudine, interferon, lamivudine, adefovir, nevirapine, delaviridine, loviride, saquinavir, indinavir, and AZT.

### BRIEF DESCRIPTION OF THE DRAWINGS

FIG. 1 is a table displaying exemplary compounds of the invention.

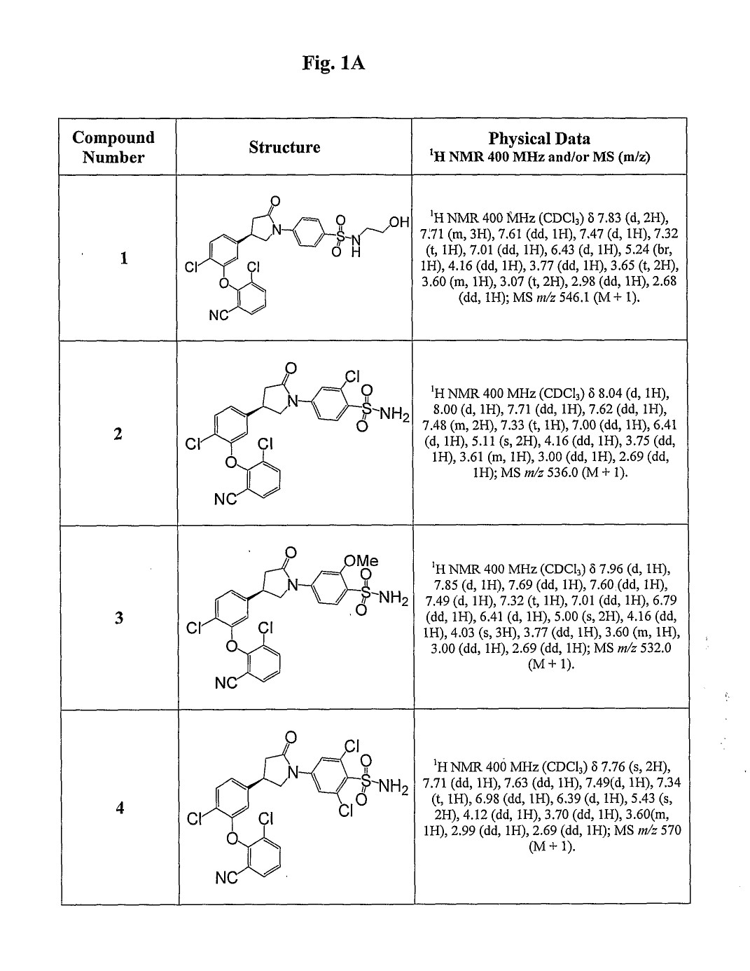

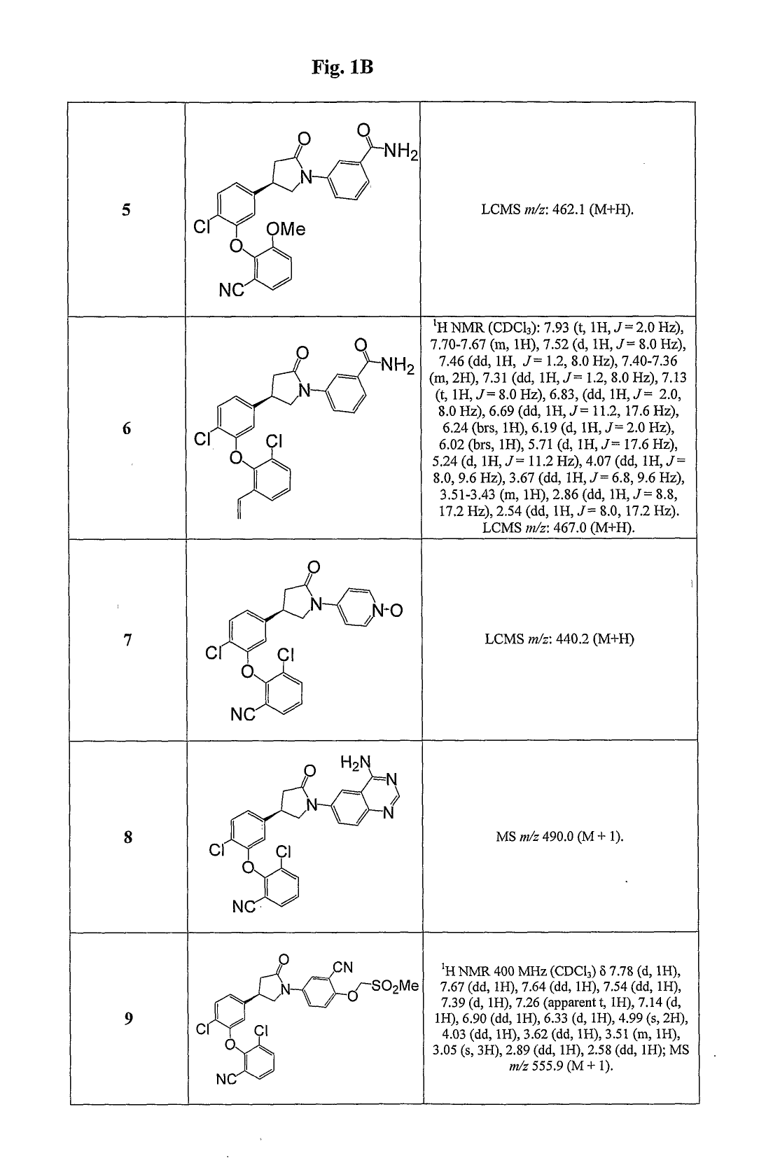

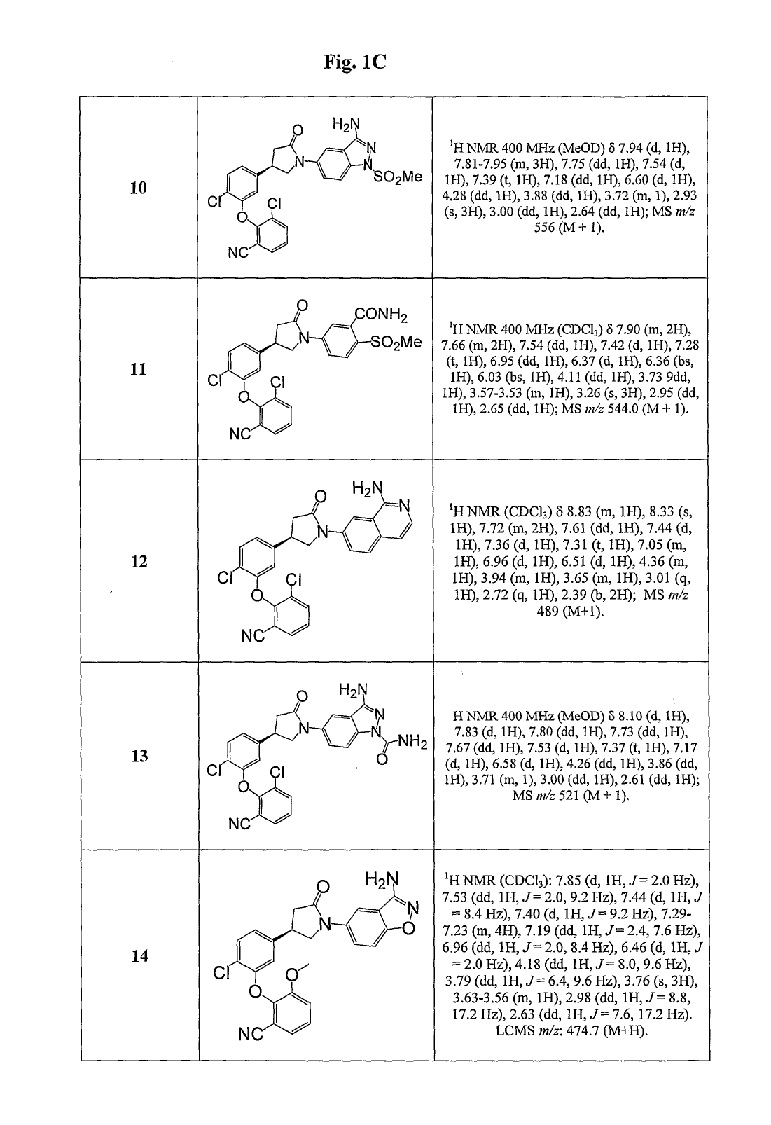

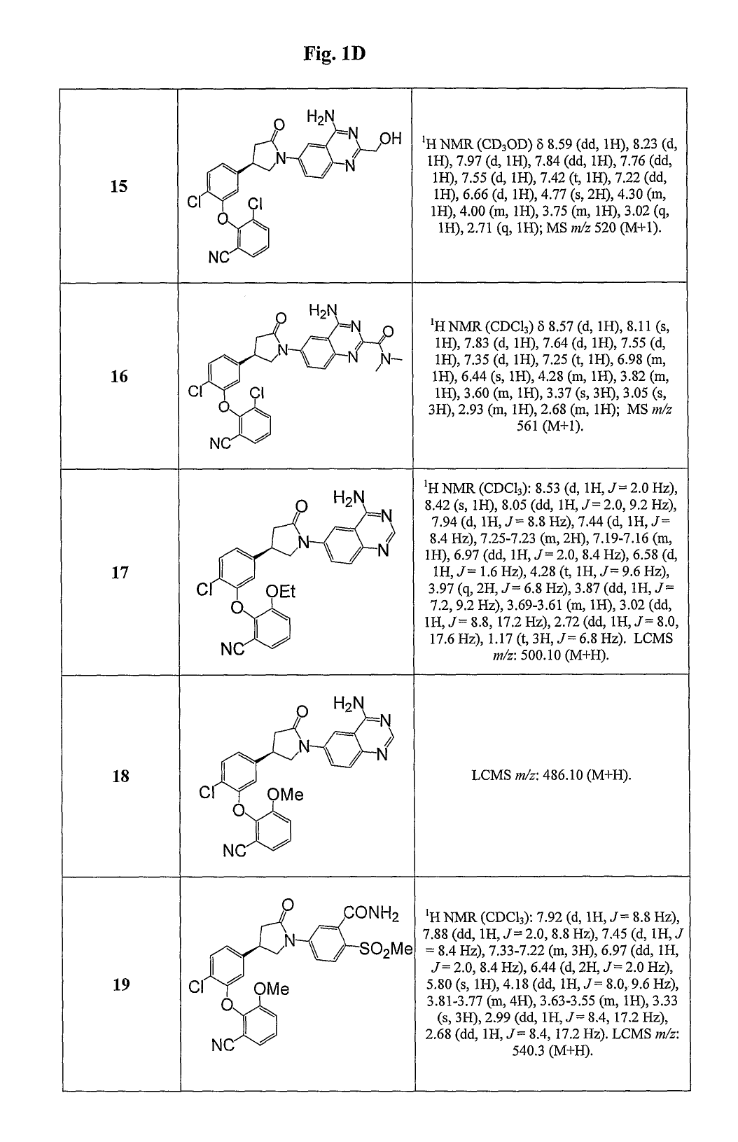

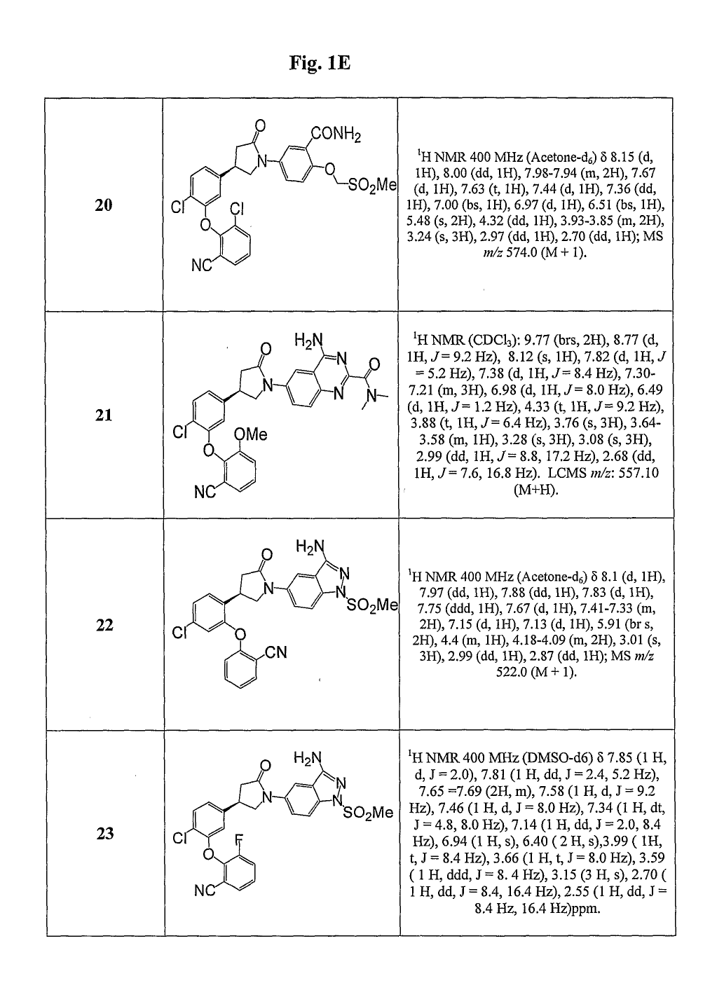

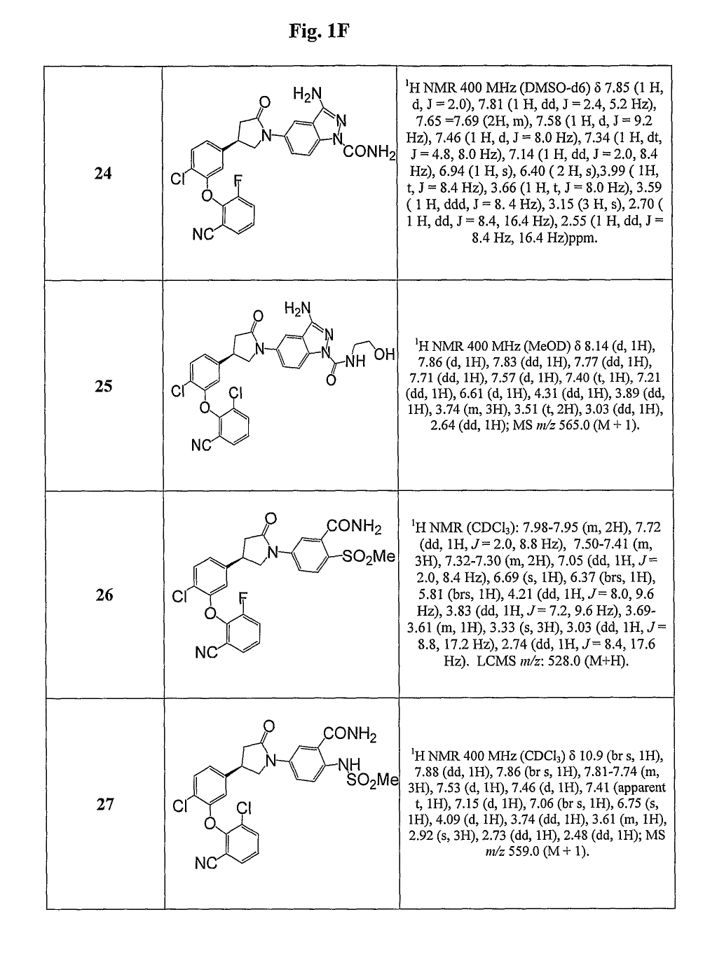

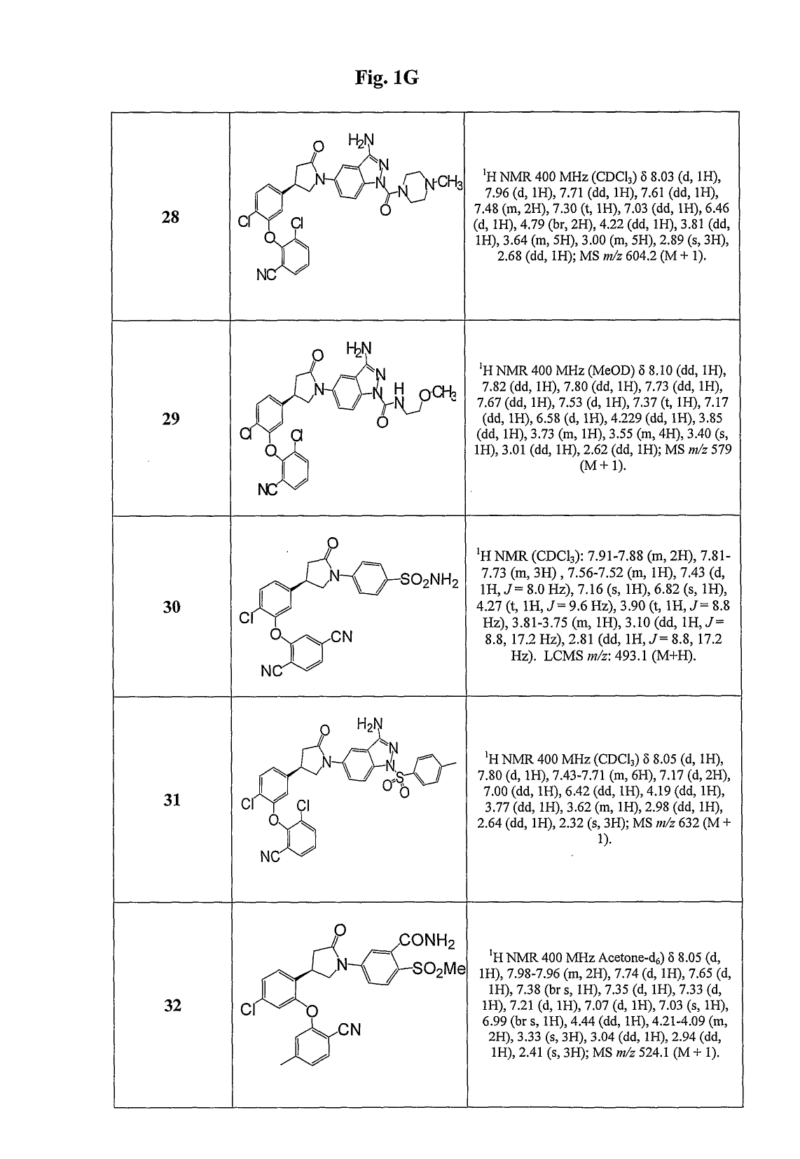

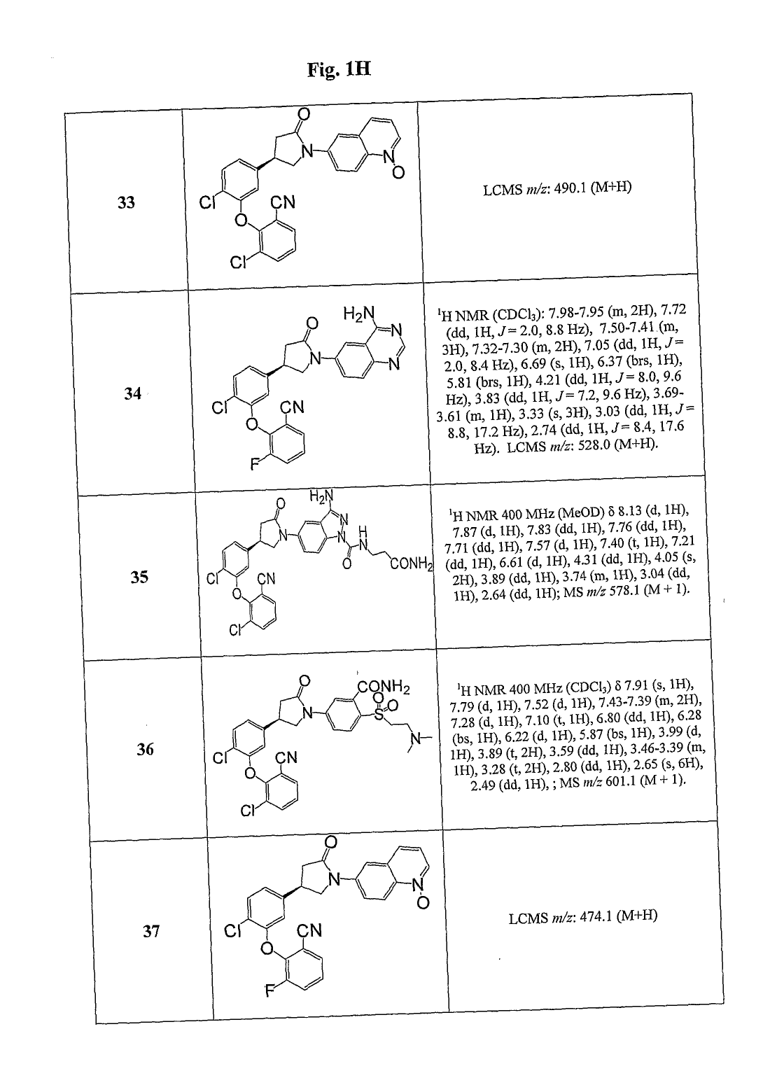

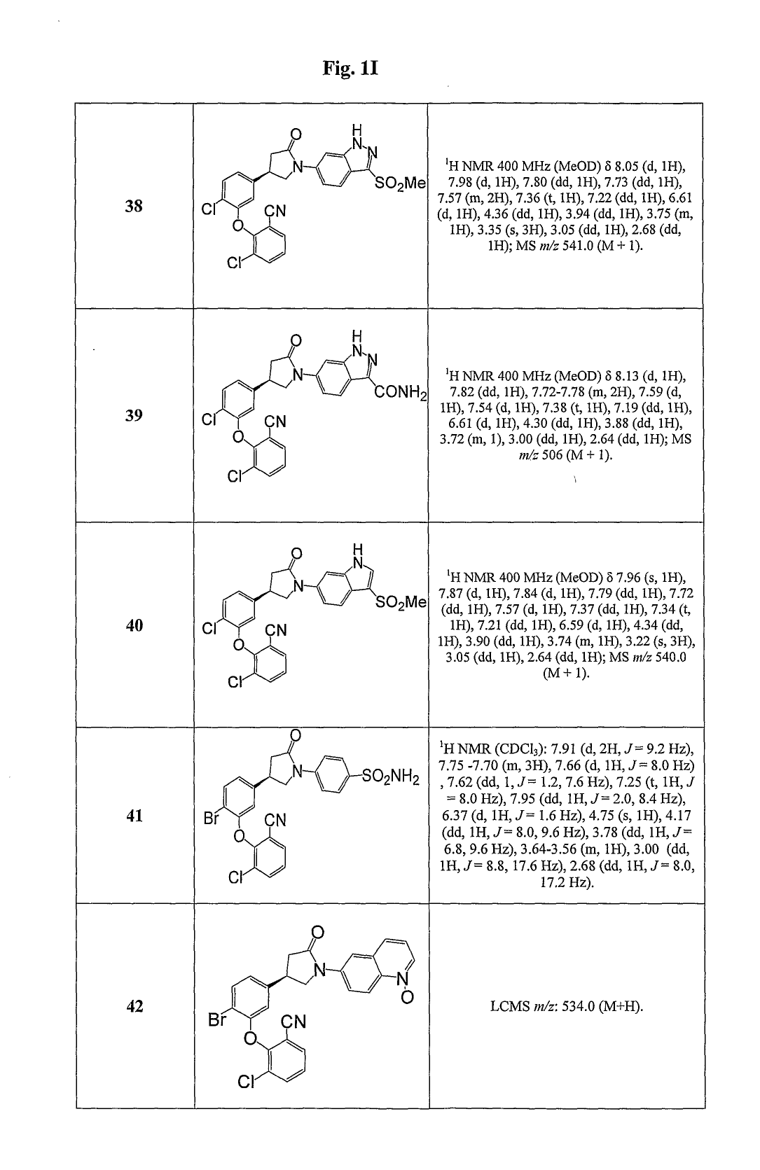

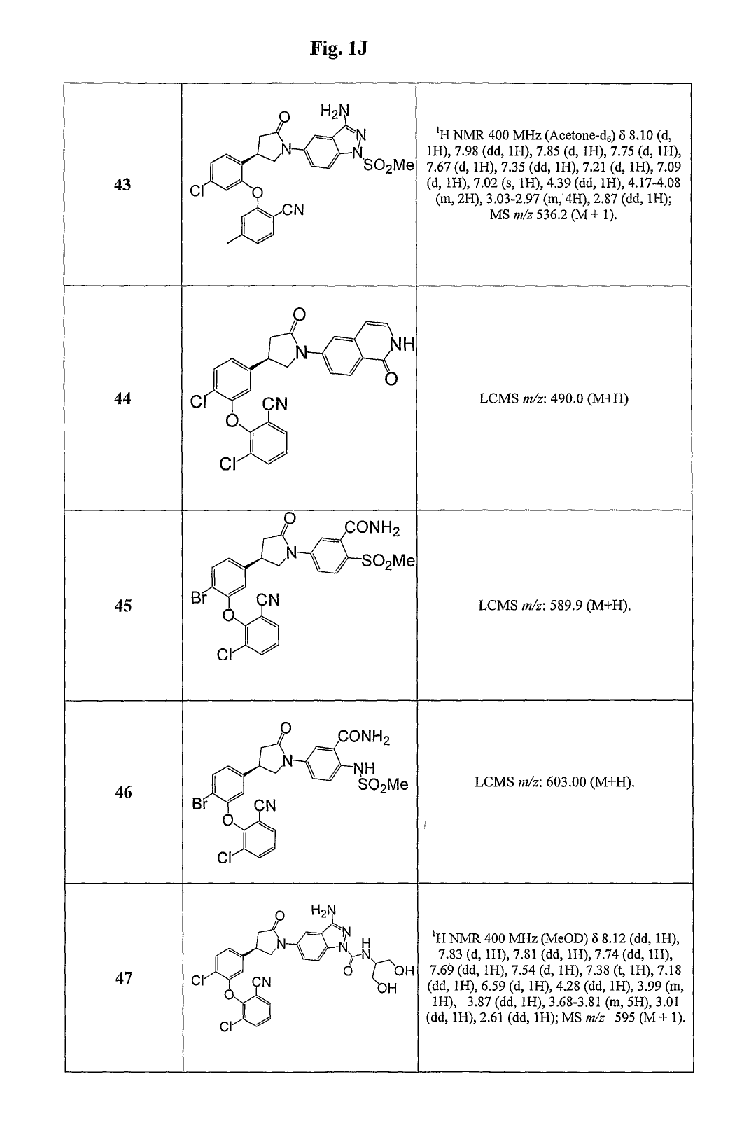

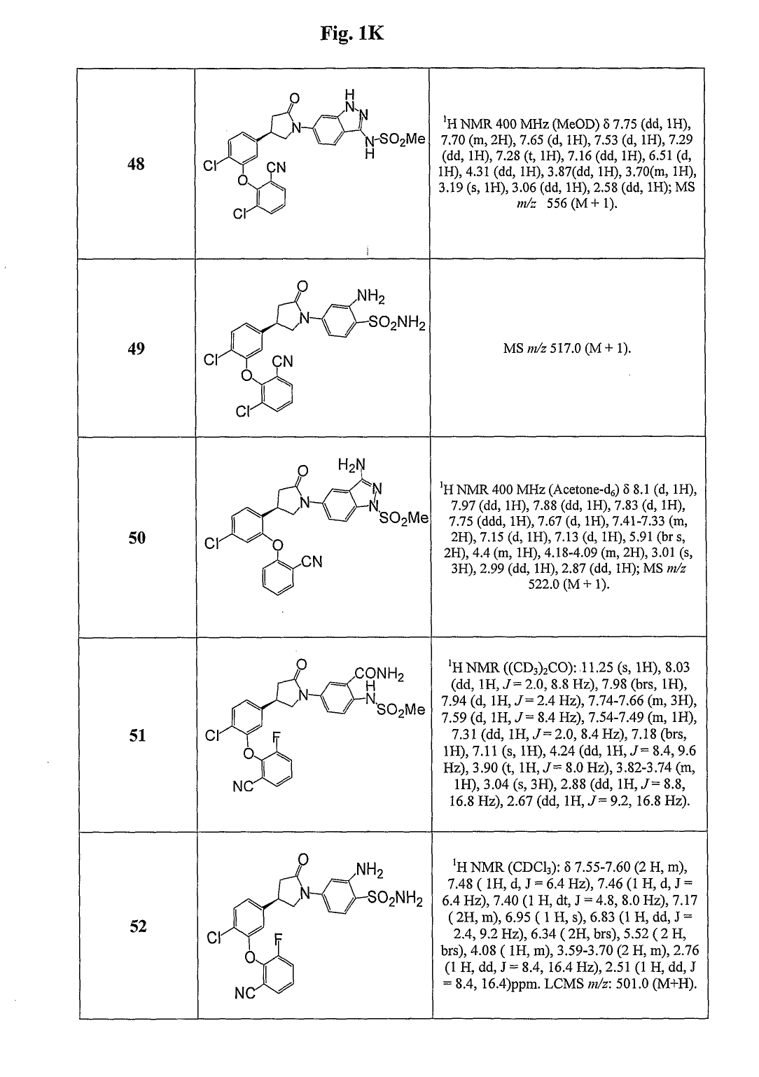

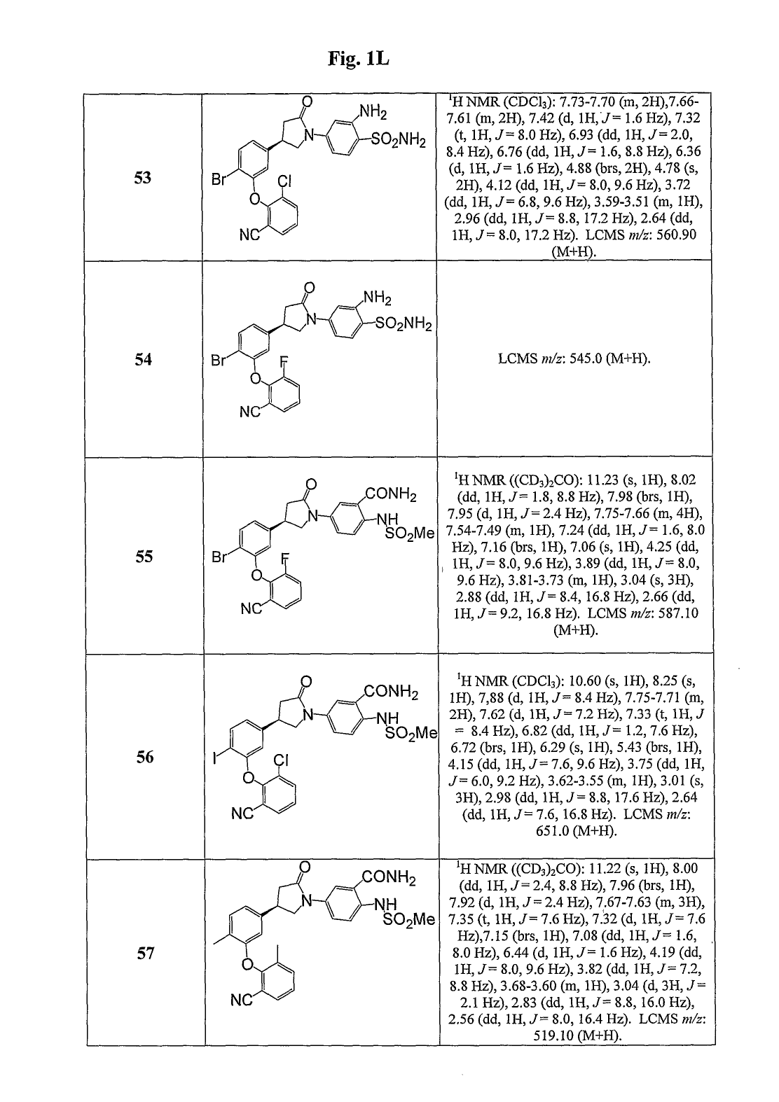

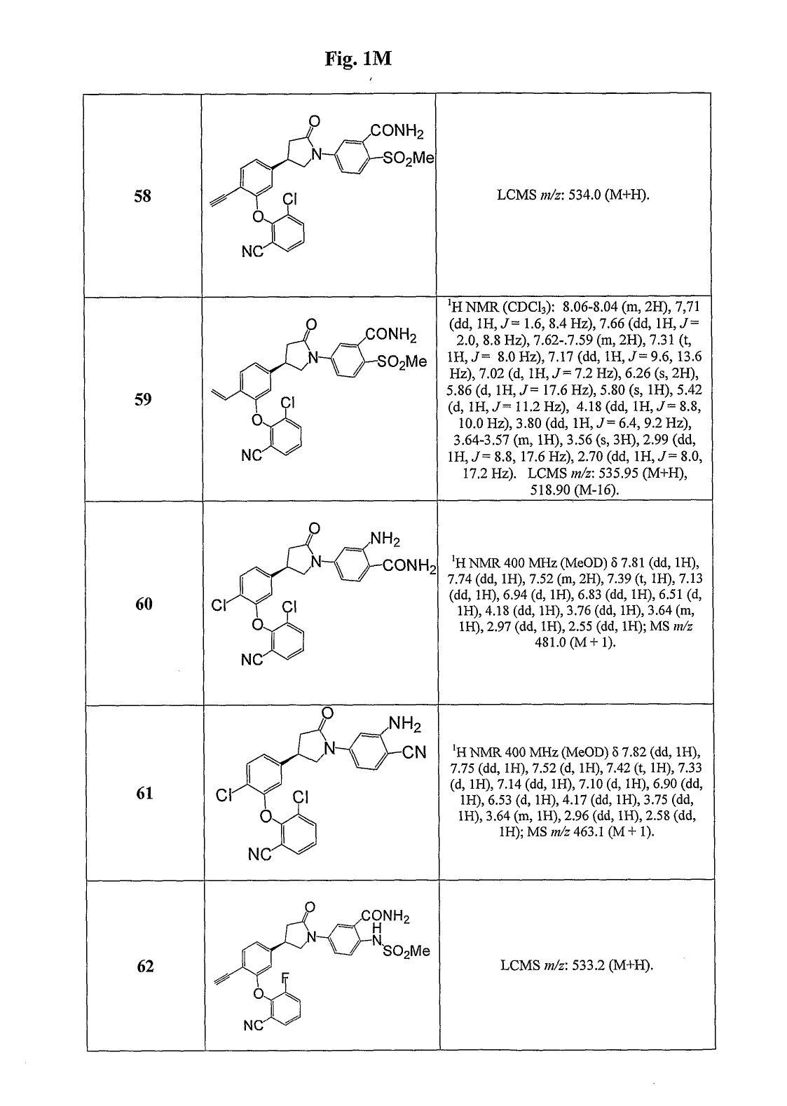

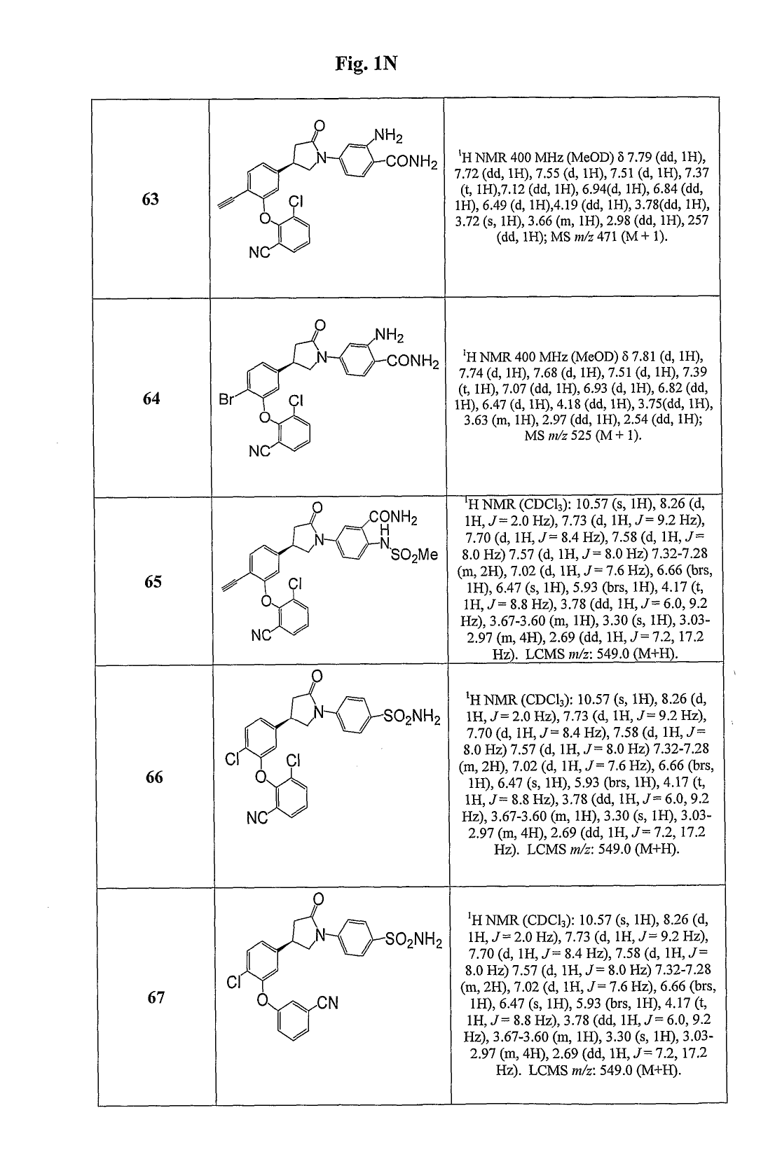

### DETAILED DESCRIPTION OF THE INVENTION AND PREFERRED EMBODIMENTS

### I. Introduction

The present invention provides new methods of preventing viral infection (e.g., HIV), killing virally infected cells (e.g., HIV infected cells) and generally inhibiting viral replication (e.g., HIV). The present invention is based, in part, on the surprising discovery that the pyrrolidones of the invention are effective to inhibit HIV infection, kill HIV infected cells and/or prevent HIV infection in the individual.

The present invention provides compounds and pharmaceutical formulations that include those compounds. Moreover, the invention also provides methods of inhibiting HIV in a cell, inhibiting reverse transcriptase in a cell, treating HIV infection in a human subject, and providing prophylaxis against HIV infection by administering at least one compound of the invention to a patient in need of such treatment.

### II. Definitions

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitrites, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, examines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

“Non-covalent protein binding groups” are moieties that interact with an intact or denatured polypeptide in an associative manner. The interaction may be either reversible or irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, thiosulfate, and thiosulfonate.

As used herein, “linking member” refers to a covalent chemical bond that includes at least one heteroatom. Exemplary linking members include —C(O)NH—, —C(O)O—, —NH—, —S—, —O—, and the like.

The term “targeting group” is intended to mean a moiety that is: (1) able to actively direct the entity to which it is attached (e.g., contrast agent) to a target region, e.g., a tumor; or (2) is preferentially passively absorbed by or entrained within a target tissue, for example a tumor. The targeting group can be a small molecule, which is intended to include both non-peptides and peptides. The targeting group can also be a macromolecule, which includes, but is not limited to, saccharides, lectins, receptors, ligand for receptors, proteins such as BSA, antibodies, poly(ethers), dendrimers, poly(amino acids) and so forth.

The term “cleavable group” is intended to mean a moiety that allows for release of the chelate from the rest of the conjugate by cleaving a bond linking the chelate (or chelate linker arm construct) to the remainder of the conjugate. Such cleavage is either chemical in nature, or enzymatically mediated. Exemplary enzymatically cleavable groups include natural amino acids or peptide sequences that end with a natural amino acid.

In addition to enzymatically cleavable sites, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available from suppliers such as Pierce.

The symbol [symbol] whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH—N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—CR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, John Wiley & Sons, New York, 1991.

By “disorder associated with HIV infection” or “disease associated with HIV infection” herein is meant a disease state which is marked by HIV infection. Such disorders associated with HIV infection include, but are not limited to, AIDS; Kaposi's sarcoma; opportunistic infections such as those caused by Pneumocystis carinii and Mycobacterium tuberculosis; oral lesions, including thrush, hairy leukoplakia, and aphthous ulcers; generalized lymphadenopathy; shingles; thrombocytopenia; aseptic meningitis; neurologic disease such as toxoplasmosis, cryptococcosis, CMV infection, primary CNS lymphoma, and HIV-associated dementia; peripheral neuropathies, seizures; and myopathy.

As used herein, “HIV reverse transcriptase inhibitor” is intended to refer to both nucleoside and non-nucleoside inhibitors of HIV reverse transcriptase (RT). Examples of nucleoside RT inhibitors include, but are not limited to, AZT, ddC, ddI, d4T, and 3TC. Examples of non-nucleoside RT inhibitors include, but are no limited to, delavirdine (Pharmacia and Upjohn U90152S), efavirenz (DuPont), nevirapine (Boehringer Ingelheim), Ro 18,893 (Roche), trovirdine (Lilly), MKC-442 (Triangle), HBY 097 (Hoechst), ACT (Korean Research Institute), UC-781 (Rega Institute), UC-782 (Rega Institute), RD4-2025 (Tosoh Co. Ltd.), and MEN 10979 (Menarini Farmaceutici).

As used herein, “HIV protease inhibitor” is intended to refer to compounds which inhibit HIV protease. Examples include, but are not limited, saquinavir (Roche, Ro31-8959), ritonavir (Abbott, ABT-538), indinavir (Merck, MK-639), amprenavir (Vertex/Glaxo Wellcome), nelfinavir (Agouron, AG-1343), palinavir (Boehringer Ingelheim), BMS-232623 (Bristol-Myers Squibb), GS3333 (Gilead Sciences), KNI-413 (Japan Energy), KNI-272 (Japan Energy), LG-71350 (LG Chemical), CGP-61755 (Ciba-Geigy), PD 173606 (Parke Davis), PD 177298 (Parke Davis), PD 178390 (Parke Davis), PD 178392 (Parke Davis), U-140690 (Pharmacia and Upjohn), and ABT-378. Additional examples include the cyclic protease inhibitors disclosed in WO93/07128, WO 94/19329, WO 94/22840, and PCT Application Number US96/03426.

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

### III. Compounds

In a first aspect, the invention provides pyrrolidones and related compounds. An exemplary compound of the invention has the formula:

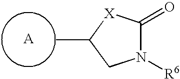

in which A represents a ring system that is selected from substituted or unsubstituted aryl and substituted or unsubstituted heteroaryl. The symbol R⁶ represents substituted or unsubstituted aryl and substituted or unsubstituted heteroaryl. X is substituted or unsubstituted carbon or substituted or unsubstituted nitrogen.

Other exemplary compounds of the invention have the formulae:

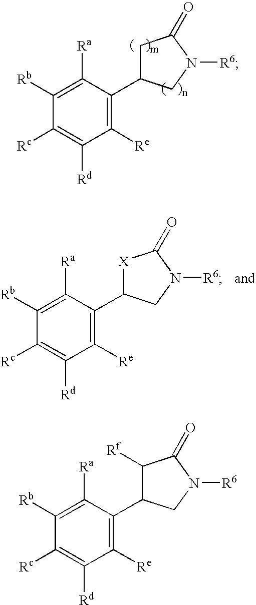

In the formulae above, the symbols m and n represent integers that are independently selected from 0, 1 and 2. X is substantially as described above.

The symbols R^{a}, R^{b}, R^{c}, R^{d}, R^{e} and R^{f} represent members independently selected from H, substituted or unsubstituted alkyl, substituted or unsubstituted cycloalkyl, substituted or unsubstituted heteroalkyl, substituted or unsubstituted aryl, substituted or unsubstituted heteroaryl, or another “alkyl substituent” as defined hereinabove. The symbol R⁶ represents substituted or unsubstituted aryl and substituted or unsubstituted heteroaryl.

In a second aspect, the invention provides a compound according to Formula I:

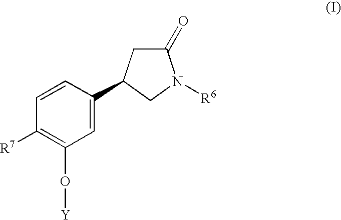

in which the symbols R⁷ and Y independently represent H, substituted or unsubstituted alkyl, substituted or unsubstituted cycloalkyl, substituted or unsubstituted heteroalkyl, substituted or unsubstituted aryl, substituted or unsubstituted heterocycloalkyl or substituted or unsubstituted heteroaryl. The symbol R⁶ represents substituted or unsubstituted aryl and substituted or unsubstituted heteroaryl.

In a third aspect, the invention provides compounds according to Formula II:

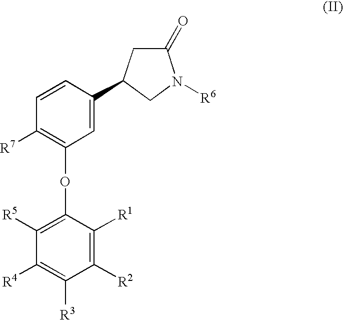

in which R¹ and R⁵ can be independently selected from H, CN, halogen, substituted or unsubstituted C₁-C₄ alkyl, substituted or unsubstituted C₂-C₄ alkenyl and OR⁸. R⁸ can be a substituted or unsubstituted C₁-C₄ alkyl and C₁-C₄ haloalkyl. R² and R⁴ can be independently selected from H, halogen, CN, and substituted or unsubstituted C₁-C₄ alkyl. R³ can be H, CN, and alkyl. R⁶ can be a fused phenyl heterocyclic ring system and:

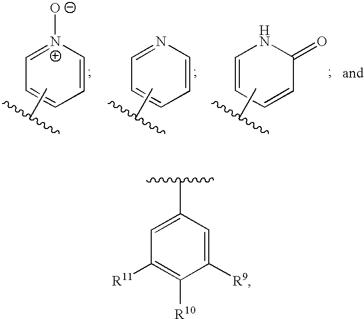

in which R⁹ and R¹¹ can be independently selected from H, substituted or unsubstituted C₁-C₄ alkyl, halogen, CN, C(O)NR¹²R¹² and NR¹²R¹² and OR¹². R¹² can be H, substituted or unsubstituted C₁-C₄ alkyl. R¹⁰ can be H, CN, NR¹³R¹⁴, SO₂NHR¹³, NHSO₂R¹³, SO₂NH(CH₂)ₙOR³, SO₂NH(CH₂)ₙNR¹³R¹⁴, O(CH₂)ₙSO₂NHR¹³, O(CH₂)ₙNR¹³R¹⁴, O(CH₂)ₙSO₂R⁵, SO₂R¹⁵, SO₂(CH₂)ₙNR¹³R¹⁴ and C(O)NR¹³R¹⁴. R¹³ and R¹⁴ can be H and substituted or unsubstituted C₁-C₄ alkyl. In addition, R¹³ and R¹⁴, together with the nitrogen to which they are attached, can be optionally joined to form a heterocyclic ring. R¹⁵ can be substituted or unsubstituted C₁-C₄ alkyl. The symbol n can be an integer from 1 to 8. In this embodiment, at least one member selected from R⁹, R¹⁰ and R¹¹ can be other than H. R⁷ can be halogen, C₁-C₄ alkyl, C₂-C₄ alkenyl and C₂-C₄ alkynyl.

In another selected embodiment, R⁶ is a fused phenyl heterocyclic ring system. This fused phenyl heterocyclic ring system can be:

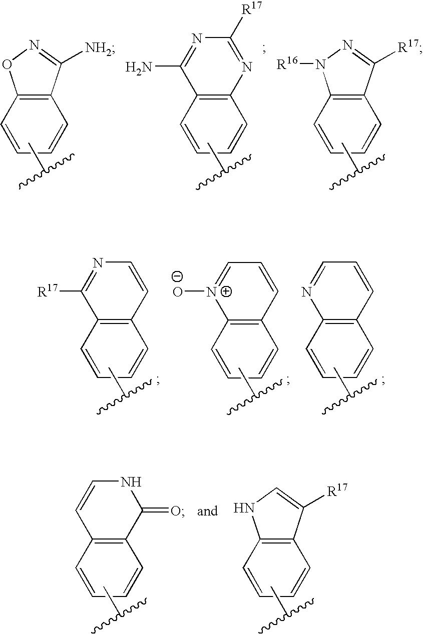

R¹⁶ can be H, C(O)NR¹⁸R¹⁹, C(O)NR¹⁸(CH₂)ₙNR¹⁸R¹⁹, C(O)NR¹⁸(CH₂)ₙOR¹⁸, C(O)NR¹⁸CH(CH₂OR¹⁸)₂, C(O)NR¹⁸(CH₂)ₙC(O)NR¹⁸R¹⁹, (CH₂)ₙSO₂NR¹⁸R¹⁹, S(O)₂R²⁰. R¹⁸ and R¹⁹ can be independently selected from H and substituted or unsubstituted C₁-C₆ alkyl. In addition, R¹⁸ and R¹⁹, together with the nitrogen atom to which they are both attached, can form a substituted or unsubstituted heterocyclic ring. The symbol n can be an integer from 1 to 8. R²⁰ can be substituted or unsubstituted C₁-C₆ alkyl and substituted or unsubstituted phenyl. R¹⁷ can be H, NH₂, (CH₂)ₘOH, C(O)NR²¹R²², SO₂R²³, NHSO₂R²³, NHCOR²³. R²¹ and R²² can be independently selected from H, substituted or unsubstituted C₁-C₆ alkyl and substituted or unsubstituted phenyl. In addition, R²¹ and R²², together with the nitrogen to which they are attached, can be optionally joined to form a ring. R²³ can be substituted or unsubstituted C₁-C₆ alkyl. The symbol m is an integer from 1 to 5.

In yet another selected embodiment, R¹ and R⁵ can be independently selected from hydrogen, halogen, CN, methyl, methoxy, vinyl and trifluoromethoxy.

In yet another selected embodiment, R⁶ can be

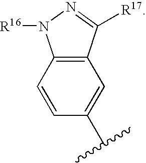

In another selected embodiment, R¹⁶ can be a member selected from C(O)NR¹⁸R¹⁹ and S(O)₂R²⁰. In another selected embodiment, R¹⁸ and R¹⁹ can be H. In another selected embodiment, R²⁰ can be CH₃. In another selected embodiment, R¹⁷ is NH₂. In another selected embodiment, R⁶ can be

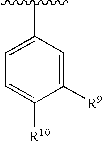

in which R⁹ can be a member selected from NH₂ and C(O)NH₂. R¹⁰ can be a member selected from C(O)NH₂, NHSO₂CH₃ and SO₂NH₂.

In another exemplary embodiment, the compound of the invention can have a formula selected from one of the following:

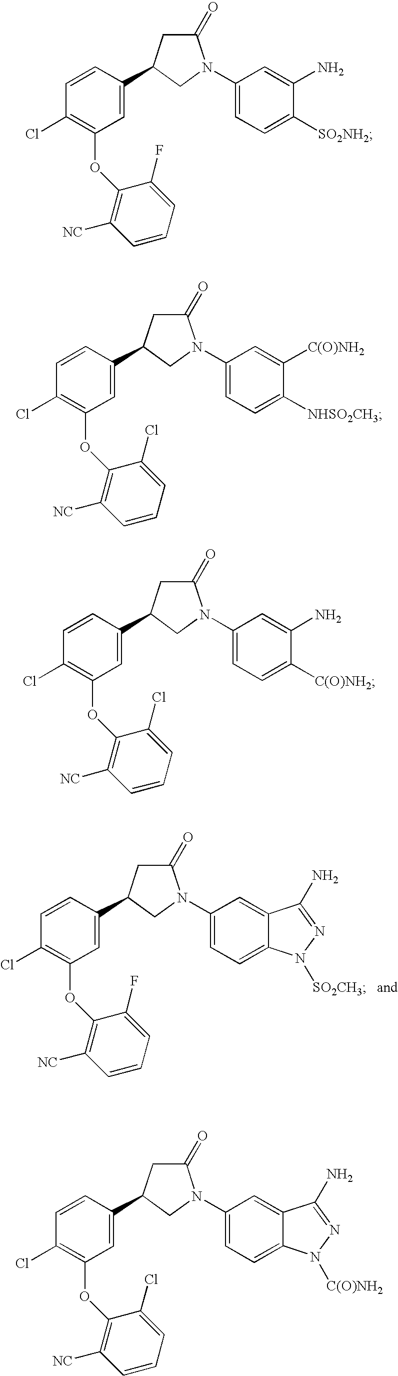

In another exemplary embodiment, at least one of the substituents is a moiety that increases the water-solubility of the parent compound. Exemplary moieties of use for increasing a compound's water solubility include ethers and polyethers, e.g., a member selected from ethylene glycol, and ethylene glycol oligomers, having a molecular weight of from about 60 daltons to about 10,000 daltons, and more preferably of from about 100 daltons to about 1,000 daltons.

Representative polyether-based substituents include, but are not limited to, the following structures:

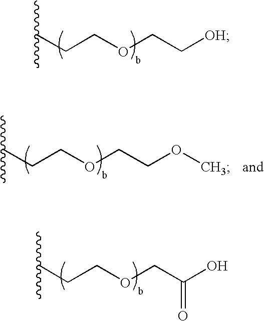

in which b is preferably a number from 1 to 100, inclusive. Other functionalized polyethers are known to those of skill in the art, and many are commercially available from, for example, Shearwater Polymers, Inc. (Alabama).

In another exemplary embodiment, at least one of R¹-R⁷ is a linker moiety that includes a reactive functional group for conjugating the compound to another molecule or to a surface. The linkers of use in the compounds of the invention can also include a cleaveable group. In an exemplary embodiment, the cleaveable group is interposed between the pyrrolidone core and a targeting agent or macromolecular backbone. Representative useful reactive groups are discussed in greater detail in succeeding sections. Additional information on useful reactive groups is known to those of skill in the art. See, for example, Hermanson, BIOCONJUGATE TECHNIQUES, Academic Press, San Diego, 1996.

### III. a) Specific Moieties on the Compounds

In this section, certain moieties on the compounds of the invention are highlighted, such as reactive functional groups, targeting agents, macromolecular complexes, polysaccharides, dendrimer-based agents, and poly(ethylene glycol)-based agents. In this section, methods of joining these moieties to the compounds of the invention are described. In these methods, a compound of the invention is reacted with a moiety to form a “conjugate” of the invention. Please note that these “conjugates” are also encompassed in the terms “compound” and “compounds of the invention” as used in the specification and claims. “Conjugate” is only used here to differentiate the reactant “compound”, which can be a “compound of the invention”, from the product, which can also be a “compound of the invention”.

III. a1) Reactive Functional Groups

As discussed above, the pyrrolidone core of the compounds of the invention are optionally tethered to other species by means of bonds formed between a reactive functional group on the pyrrolidone or a linker attached to the pyrrolidone, and a reactive functional group of complementary reactivity on the other species. For clarity of illustration the succeeding discussion focuses on the conjugation of representative pyrrolidones of the invention to polymers, including poly(ethers) and dendrimers, and to targeting agents useful for translocating the pyrrolidone-targeting agent conjugate across a membrane. The focus exemplifies selected embodiments of the invention from which others are readily inferred by one of skill in the art. No limitation of the invention is implied by focusing the discussion on the representative embodiments.

Exemplary pyrrolidones of the invention bear a reactive functional group, which is generally located on the pyrrolidone ring or on a substituted or unsubstituted alkyl or heteroalkyl chain attached to the ring, allowing their facile attachment to another species. A convenient location for the reactive group is the terminal position of an alkyl or heteroalkyl substituent of the pyrrolidone core.

Reactive groups and classes of reactions useful in practicing the present invention are generally those that are well known in the art of bioconjugate chemistry. Currently favored classes of reactions available with reactive analogues are those proceeding under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Exemplary reactive groups include the reaction of carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters. Hydroxyl groups can be converted to esters, ethers, aldehydes, etc. Haloalkyl groups are converted to new species by reaction with, for example, an amine, a carboxylate anion, thiol anion, carbanion, or an alkoxide ion. Dienophile (e.g., maleimide) groups participate in Diels-Alder. Aldehyde or ketone groups can be converted to imines, hydrazones, semicarbazones or oximes, or via such mechanisms as Grignard addition or alkyllithium addition. Sulfonyl halides react readily with amines, for example, to form sulfonamides. Amine or sulfhydryl groups are, for example, acylated, alkylated or oxidized. Alkenes, can be converted to an array of new species using cycloadditions, acylation, Michael addition, etc. Epoxides react readily with amines and hydroxyl compounds.

Exemplary combinations of reactive functional groups found on a compound of the invention and on a targeting moiety (or polymer or linker) are set forth in Table 1. If the reactive functional group is chemical functionality 1, then the targeting moiety is chemical functionality 2, and vice versa.

TABLE 1

| Chemical | Chemical | |
|---|---|---|
| Functionality 1 | Functionality 2 | Linkage |
| Hydroxy | Carboxy | Ester |
| | Hydroxy | Carbonate |
| | Amine | Carbamate |
| | SO₃ | Sulfate |
| | PO₃ | Phosphate |
| | Carboxy | Acyloxyalkyl |
| | Ketone | Ketal |
| | Aldehyde | Acetal |
| | Hydroxy | Anhydride |
| Mercapto | Mercapto | Disulfide |
| | Carboxy | Acyloxyalkyl |
| | | Thioether |
| | Carboxy | Thioester |
| | Carboxy | Amino amide |
| | Mercapto | Thioester |
| | Carboxy | Acyloxyalkyl |
| | | Ester |
| | Carboxy | Acyloxyalkyl |
| | | Amide |
| | Amino | Acyloxyalkoxy |
| | | Carbonyl |
| | Carboxy | Anhydride |
| | Carboxy | N-acylamide |
| | Hydroxy | Ester |
| | Hydroxy | Hydroxymethyl |
| | | ketone ester |
| | Hydroxy | Alkoxycarbonyl |
| | | oxyalkyl |
| Amino | Carboxy | Acyloxyalkylamine |
| | Carboxy | Acyloxyalkylamide |
| | Amino | Urea |
| | Carboxy | Amide |
| | Carboxy | Acyloxyalkoxycarbonyl |
| | Amide | N-Mannich base |
| | Carboxy | Acyloxyalkyl carbamate |
| Phosphate | Hydroxy | Phosphate |
| Oxygen ester | Amine | Phosphoramidate |
| | Mercapto | Thiophosphate ester |
| Ketone | Carboxy | Enol ester |
| Sulfonamide | Carboxy | Acyloxyalkyl sulfonamide |
| | Ester | N-sulfonyl-imidate |

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, PRODRUGS: TOPICAL AND OCULAR DRUG DELIVERY, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates.

The reactive functional groups can be chosen such that they do not participate in, or interfere with, the reactions necessary to assemble the reactive ligand analogue. Alternatively, a reactive functional group can be protected from participating in the reaction by the presence of a protecting group. Those of skill in the art will understand how to protect a particular functional group from interfering with a chosen set of reaction conditions. For examples of useful protecting groups, see Greene et al., PROTECTIVE GROUPS IN ORGANIC SYNTHESIS, John Wiley & Sons, New York, 1991.

Generally, prior to forming the linkage between the ligand and the targeting (or other) agent, and optionally, the linker group, at least one of the chemical functionalities will be activated. One skilled in the art will appreciate that a variety of chemical functionalities, including hydroxy, amino, and carboxy groups, can be activated using a variety of standard methods and conditions. For example, a hydroxyl group of the ligand (or targeting agent) can be activated through treatment with phosgene to form the corresponding chloroformate, or p-nitrophenylchloroformate to form the corresponding carbonate.

In an exemplary embodiment, the invention makes use of a targeting agent that includes a carboxyl functionality. Carboxyl groups may be activated by, for example, conversion to the corresponding acyl halide or active ester. This reaction may be performed under a variety of conditions as illustrated in March, supra pp. 388-89. In an exemplary embodiment, the acyl halide is prepared through the reaction of the carboxyl-containing group with oxalyl chloride. The activated agent is combined with a ligand or ligand-linker arm combination to form a conjugate of the invention. Those of skill in the art will appreciate that the use of carboxyl-containing targeting agents is merely illustrative, and that agents having many other functional groups can be conjugated to the ligands of the invention.

III. a2) Targeting Agents

The compounds of the invention may also be conjugated to an agent that targets the compound to a specific tissue or region of disease. Targeting agents include species that are taken up by cells using either active or passive mechanisms.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:1 4255-14258 (1995)).

Examples of peptide sequences include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically linked to the compounds of the invention.

Such subsequences can be used to translocate compounds of the invention across a cell membrane. Compounds of the invention can be conveniently fused to or derivatized with such sequences. Typically, the translocation sequence is provided as part of a fusion protein. Optionally, a linker as described herein can be used to link the compound of the invention and the translocation sequence. Any suitable linker can be used, e.g., a peptide linker or other chemical linkers.

Toxin molecules also have the ability to transport compounds across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun., 61:5147-5156 (1993); Stemnark et al., J. Cell Biol. 113:1025-1032 (1991); Donnelly et al., PNAS 90:3530-3534 (1993); Carbonetti et al., Abstr. Annu. Meet. Am. Soc. Microbiol. 95:295 (1995); Sebo et al., Infect. Immun. 63:3851-3857 (1995); Klimpel et al., PNAS U.S.A. 89:10277-10281 (1992); and Novak et al., J. Biol. Chem. 267:17186-17193 1992)).

Non-covalent protein binding groups are also of use to target the compounds of the invention to specific regions of the body and to increase the half-life of the agent through protein binding.

III. a3) Macromolecular Complexes

In an exemplary embodiment, the invention provides a macromolecular, i.e., MW>1000 D, conjugate between the pyrrolidone core and a macromolecular species. In one embodiment, a macromolecular conjugate of the invention is formed by covalently conjugating a pyrrolidone to a macromolecule via a reactive functional group. In another embodiment, the macromolecular complex is formed by a non-covalent interaction between a pyrrolidone derivative and a macromolecule, e.g, a serum protein.

In the following discussion, the invention is described by reference to specific macromolecules of use for forming conjugates with the novel pyrrolidone cores of the invention. Those of skill in the art will appreciate that the focus of the discussion is for clarity of illustration and does not limit the scope of the invention. The invention provides macromolecular conjugates that include components derived from biomolecules and synthetic molecules. Exemplary biomolecules include polypeptides (e.g., antibodies, enzymes, receptors, antigens); polysaccharides (e.g., starches, inulin, dextran); lectins, non-peptide antigens and the like. Exemplary synthetic polymers include poly(acrylic acid), poly(lysine), poly(glutamic acid), poly(ethylene imine), etc.

III. a3i) Covalent Conjugation of the Macromolecular Complexes

Selection of an appropriate reactive functional group on a pyrrolidone core of the invention to form a desired macromolecular species is well within the abilities of one of skill in the art. Exemplary reactive functional groups of use in forming the covalent conjugates of the invention are discussed above. It is well within the abilities of one of skill to select and prepare a pyrrolidone core of the invention having an appropriate reactive functional group of complementary reactivity to a reactive group on its conjugation partner.

In one embodiment, the bond formed between reactive functional groups of the macromolecule and that of the pyrrolidone attaches the pyrrolidone to the macromolecule essentially irreversibly via a “stable bond” between the components. A “stable bond”, as used herein, is a bond, which maintains its chemical integrity over a wide range of conditions (e.g., amide, carbamate, carbon-carbon, ether, etc.). In another embodiment, the macromolecule and the pyrrolidone are linked by a “cleaveable bond”. A “cleaveable bond”, as used herein, is a bond that undergoes scission under selected conditions. Cleaveable bonds include, but are not limited to, disulfide, imine, carbonate and ester bonds. As discussed in the preceding sections, the reactive functional group can be located at one or more positions of the pyrrolidone.

III. a4) Polysaccharides

In an exemplary embodiment, the present invention provides conjugates between a pyrrolidone core and saccharides, e.g., polysaccharides. In an exemplary embodiment, the invention provides a conjugate between an oxygen donor chelate and inulin. Inulin is a naturally occurring polysaccharide which has been previously investigated as a carrier for diagnostic moieties (Rongved, P. K., J. Carbohydr. Res. 1991, 214, 315; Corsi, D. M. V. E. et al., Chem. Eur. J. 2001, 7, 64). The structure of inulin can be described as a mixture of linear β-(2→1)-linked α-D-fructofuranosyl chains with a α-D-glucopyranosyl unit at the terminal end. Inulin is commercially available in a variety of molecular weights and the degree of polymerization varies from 10 to 30, resulting in a molecular weight distribution of 1500 to 5000 Da. The high hydrophilicity, pH stability, low solution viscosity and biocompatability of inulin ensures that its conjugates have favorable pharmacological properties.

III. a5) Dendrimer-Based Agents

In another aspect, the present invention provides a pyrrolidone as set forth above, which is attached to a dendrimer via a reactive functional group. Similar to the polymeric group discussed above, the dendrimer has at least two reactive functional groups. In one embodiment, one or more formed pyrrolidone is attached to the dendrimer. Alternatively, the pyrrolidone is formed directly on the dendrimer.

In an exemplary embodiment, a water-soluble and bio-adapted polyester (polypropionate) class of dendrimer contrast agents has been designed to provide favorable pharmacokinetic properties. See, for example, Ihre, H. et al., Macromolecules 1998, 31, 4061; Ihre, H. et al., J. Am. Chem. Soc. 1996, 118, 6388; Anders, H., Ihre, H., Patent WO/9900440 (Sweden)). In an exemplary embodiment, the termini of the dendrimers are conjugated to a pyrrolidone core of the invention.

III. a6) Poly(ethylene glycol)-Based Agents

In another exemplary embodiment, the invention provides a conjugate between a pyrrolidone core of the invention and poly(ethylene glycol). Poly(ethylene glycol) (PEG) is used in biotechnology and biomedical applications. The use of this agent has been reviewed (POLY(ETHYLENE GLYCOL) CHEMISTRY: BIOTECHNICAL AND BIOMEDICAL APPLICATIONS, J. M. Harris, Ed., Plenum Press, New York, 1992). Modification of enzymes (Chiu et al., J. Bioconjugate Chem., 4: 290-295 (1993)), RGD peptides (Braatz et al., Bioconjugate Chem., 4: 262-267 (1993)), liposomes (Zalipsky, S. Bioconjugate Chem., 4: 296-299 (1993)), and CD4-IgG glycoprotein (Charnow et al., Bioconjugate Chem., 4: 133-140 (1993)) are some of the recent advances in the use of polyethylene glycol. Surfaces treated with PEG have been shown to resist protein deposition and have improved resistance to thrombogenicity when coated on blood contacting biomaterials (Merrill, “Poly(ethylene oxide) and Blood Contact: A Chronicle of One Laboratory,” in POLY(ETHYLENE GLYCOL) CHEMISTRY: BIOTECHNICAL AND BIOMEDICAL APPLICATIONS, Harris, Ed., Plenum Press, New York, (1992), pp. 199-220).

Many routes are available for attaching a pyrrolidone core of the invention onto a polymeric or oligomeric species. See, for example, Dunn, R. L., et al., Eds. POLYMERIC DRUGS AND DRUG DELIVERY SYSTEMS, ACS Symposium Series Vol. 469, American Chemical Society, Washington, D.C. 1991; Herren et al., J. Colloid and Interfacial Science 115: 46-55 (1987); Nashabeh et al., J. Chromatography 559: 367-383 (1991); Balachandar et al., Langinuir 6: 1621-1627 (1990); and Burns et al., Biomaterials 19: 423-440 (1998).

Many activated derivatives of poly(ethyleneglycol) are available commercially and in the literature. It is well within the abilities of one of skill to choose, and synthesize if necessary, an appropriate activated PEG derivative with which to prepare a conjugate useful in the present invention. See, Abuchowski et al. Cancer Biochem. Biophys., 7: 175-186 (1984); Abuchowski et al., J. Biol. Chem., 252: 3582-3586 (1977); Jackson et al., Anal. Biochem., 165: 114-127 (1987); Koide et al., Biochem Biophys. Res. Commun., 111: 659-667 (1983)), tresylate (Nilsson et al., Methods Enzymol., 104: 56-69 (1984); Delgado et al., Biotechnol. Appl. Biochem., 12: 119-128 (1990)); N-hydroxysuccinimide derived active esters (Buckmann et al., Makromol. Chem., 182: 1379-1384 (1981); Joppich et al., Makromol. Chem., 180: 1381-1384 (1979); Abuchowski et al., Cancer Biochem. Biophys., 7: 175-186 (1984); Katre et al. Proc. Natl. Acad. Sci. U.S.A., 84: 1487-1491 (1987); Kitamura et al., Cancer Res., 51: 4310-4315 (1991); Boccu et al., Z. Naturforsch., 38C: 94-99 (1983), carbonates (Zalipsky et al., POLY(ETHYLENE GLYCOL) CHEMISTRY: BIOTECHNICAL AND BIOMEDICAL APPLICATIONS, Harris, Ed., Plenum Press, New York, 1992, pp. 347-370; Zalipsky et al., Biotechnol. Appl. Biochem., 15: 100-114 (1992); Veronese et al., Appl. Biochem. Biotech., 11: 141-152 (1985)), imidazolyl formates (Beauchamp et al., Anal. Biochem., 131: 25-33 (1983); Berger et al., Blood, 71: 1641-1647 (1988)), 4-dithiopyridines (Woghiren et al., Bioconjugate Chem., 4: 314-318 (1993)), isocyanates (Byun et al., ASAIO Journal, M649-M-653 (1992)) and epoxides (U.S. Pat. No. 4,806,595, issued to Noishiki et al., (1989). Other linking groups include the urethane linkage between amino groups and activated PEG. See, Veronese, et al., Appl. Biochem. Biotechnol., 11: 141-152 (1985).

### IV. Synthesis and Purification of Pyrrolidones

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention, it is not intended to define the scope of reactions or reaction sequences that are useful in preparing the compounds of the present invention.

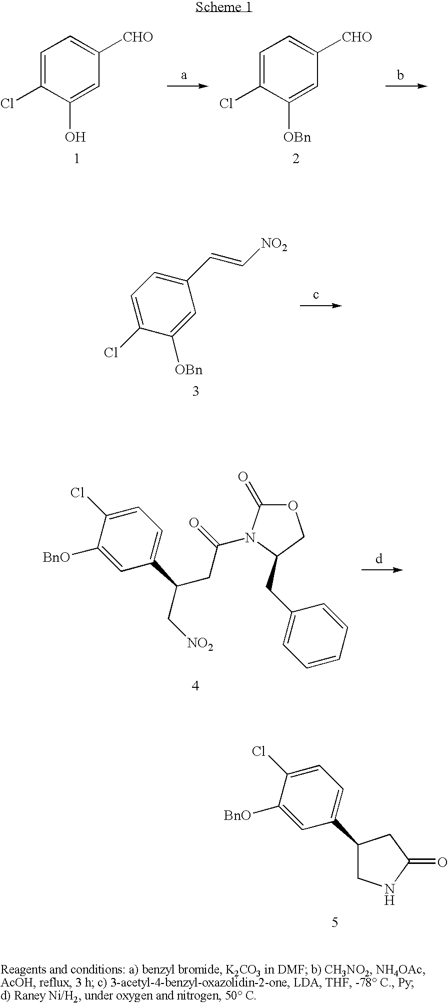

One method of synthesizing compounds of the invention is shown in Scheme 1. In this Scheme, hydroxybenzaldehyde is reacted with a benzyl group in order to provide 2 (reaction a). 2 is then reacted with a nitrate in order to provide 3. 3 is next reacted with an oxazolidinone in order to provide 4. Reduction of 4 with Raney Nickel induces an intramolecular cyclization which produces 5, a lactam comprising a 3,4-substituted phenyl group.

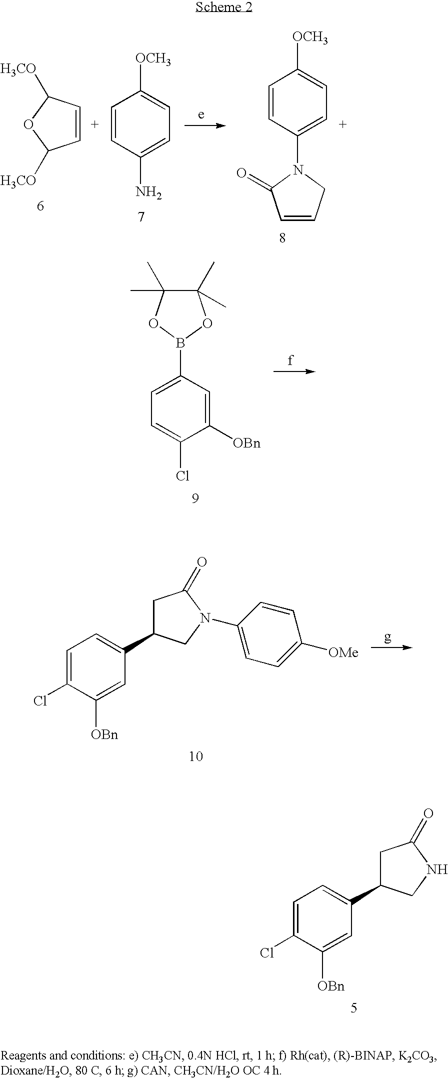

Another exemplary route to compounds of the invention is set forth in Scheme 2, which illustrates the synthesis of a lactam 5, comprising a 3,4-substituted phenyl group. A dihydrofuran 6 is reacted with p-anisidine 7 in order to provide 8 (reaction e). 8 is next reacted with borate 9 in order to provide 10 (reaction f). Finally, the N-phenyl group on 10 is removed in order to produce 5 (reaction g).

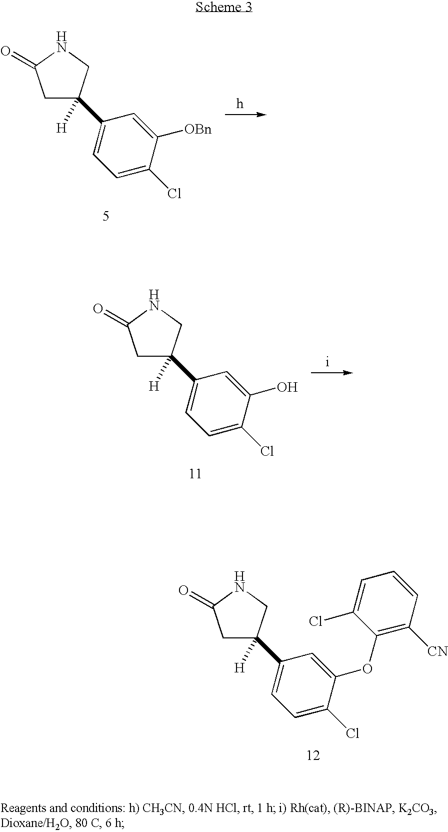

5 is further functionalized according to Scheme 3. The benzyl protecting group on 5 is first removed in order to provide 11 (reaction h). 11 is then coupled to a substituted phenyl group in order to provide 12 (reaction i).

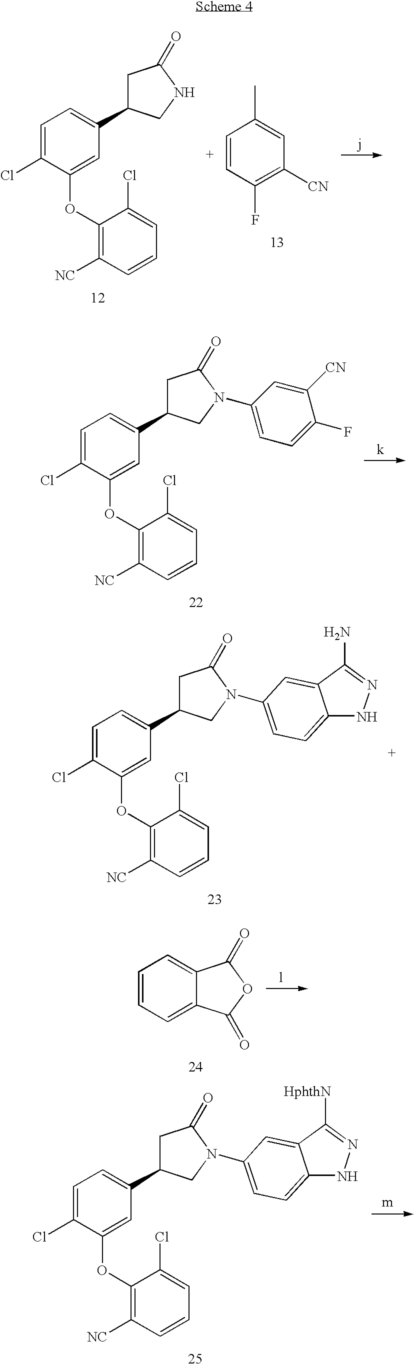

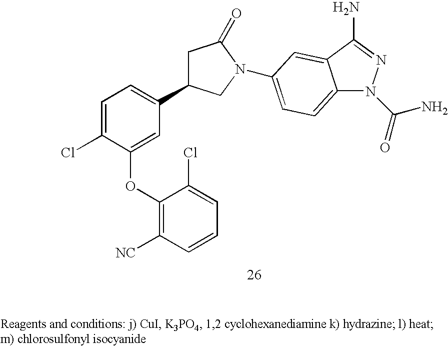

In further exemplary syntheses, the nitrogen of the lactam final product in Schemes 1-3 may be derivatized as shown in Schemes 4-9. In Scheme 4, the synthesis begins with 12. This compound is reacted with an iodo-substituted aryl group in order to produce 22 (reaction j). 22 is then reacted with hydrazine in order to produce the fused ring N-pyrrolidone compound 23. A protecting group, such as phthalic anhydride, is then added to the free amine group on 23 in order to afford 25. Removal of the protecting group then affords 26 (reaction m).

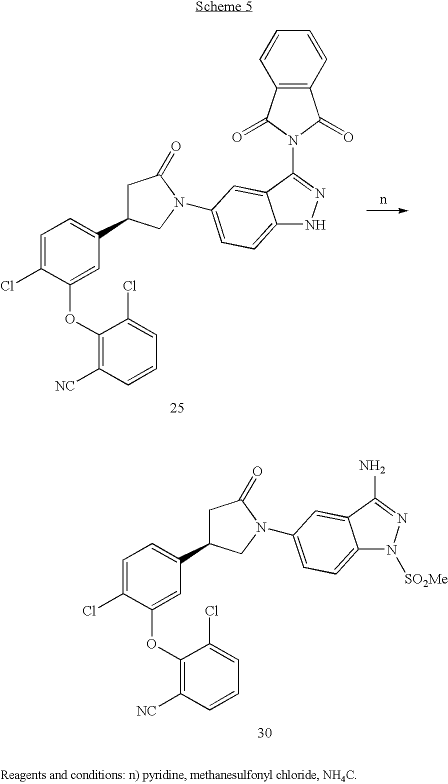

Other groups, aside from ureas, can be formed with the endocyclic nitrogen in 25. An example of this type of reaction in shown in Scheme 5. Here, 25 is reacted with a sulfonyl chloride in order to produce 30. One of skill in the art will recognize that a wide variety of N-substituted lactam derivatives may be obtained using aryl halides variously substituted with, for example, alkyls, heteroaryls, arylalkyls, and heterocycloalkyls.

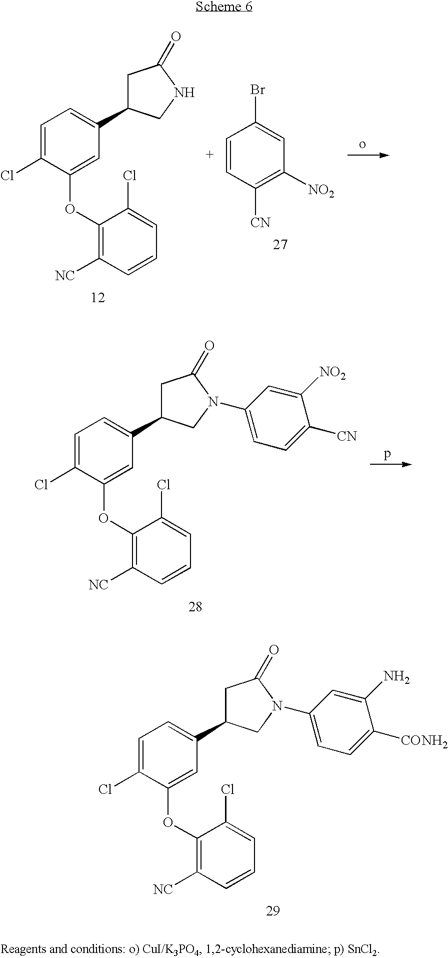

Further exemplary N-substitutions of the lactam product from Scheme 3 (though applicable to Scheme 1 and Scheme 2 products) is shown in Scheme 6. 12 is reacted with a halosubstituted phenyl group 27 in order to produce 28 (reaction o). 28 can then be subjected to various reactions in order to functionalize the newly added phenyl ring. An example of this functionalization is shown by the conversion of 28 to 29 (reaction p).

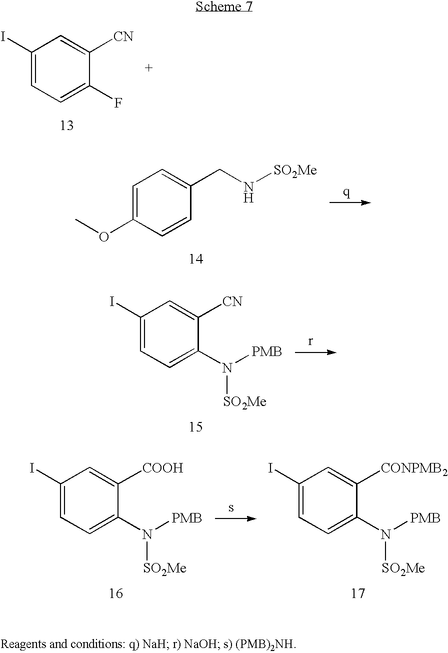

In order to synthesize some compounds of the invention, some components must be synthesized separated and then reacted with the pyrrolidone. Examples of this strategy are provided in Schemes 7-10. In Scheme 7, compound 13 and compound 14 are reacted under conditions q in order to produce compound 15. Subsequent hydride reduction affords compound 16 (reaction r). The carboxylate group on 16 is then protected under the conditions of reaction s in order to produce compound 17.

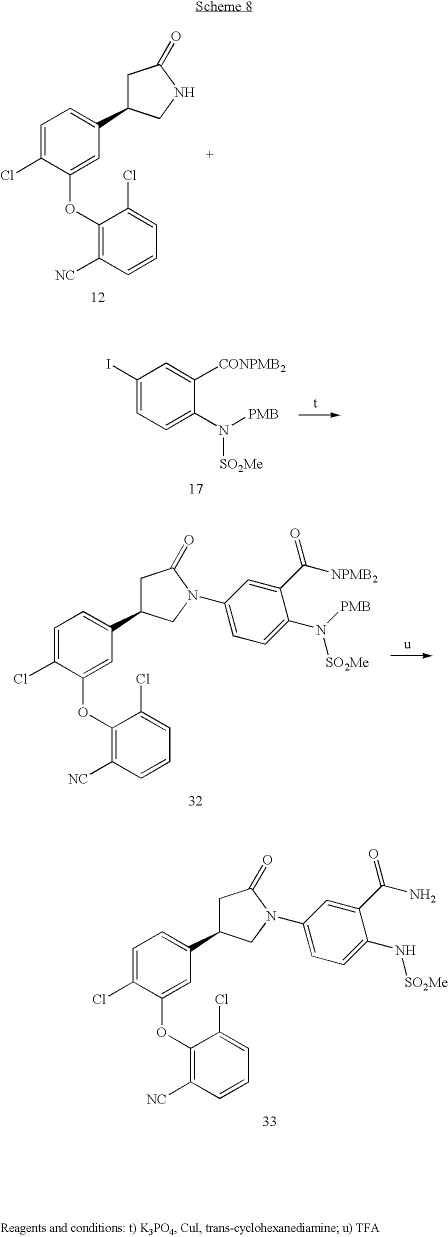

Scheme 8 illustrates another method of functionalizing the nitrogen of the lactam product from Scheme 3 (This is also applicable to the products of Scheme 1). Here, protected bromo or iodo substituted compound 17 is added to 12 in order to produce compound 32 (reaction t). The protecting groups on compound 32 are subsequently removed to provide compound 33 (reaction u).

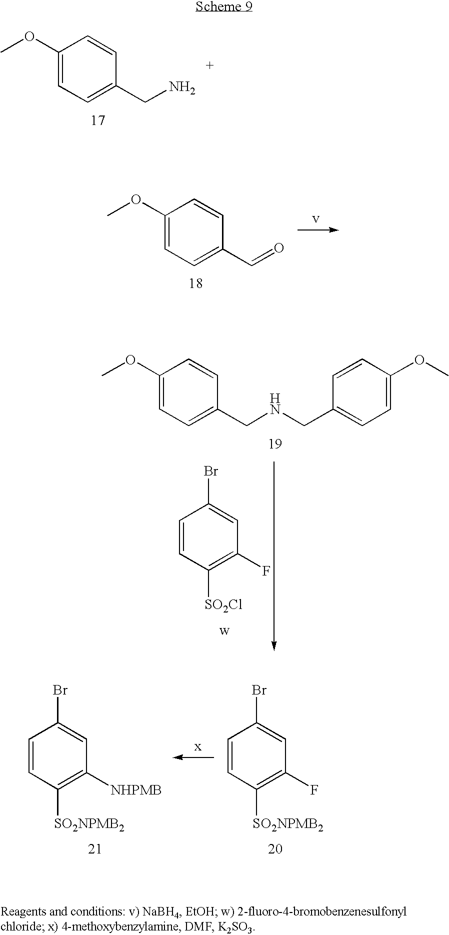

A variation on the methods of Scheme 7 is shown in Scheme 9. Here, compound 17 and compound 18 are reacted under condition v in order to produce protecting group 19. 19 is then reacted with a sulfate or sulfone substituted phenyl compound in order to protect the sulfur-comprising group 16 (reaction w). Compound 20 is then further reacted with an amine group in order to produce 21 (reaction x).

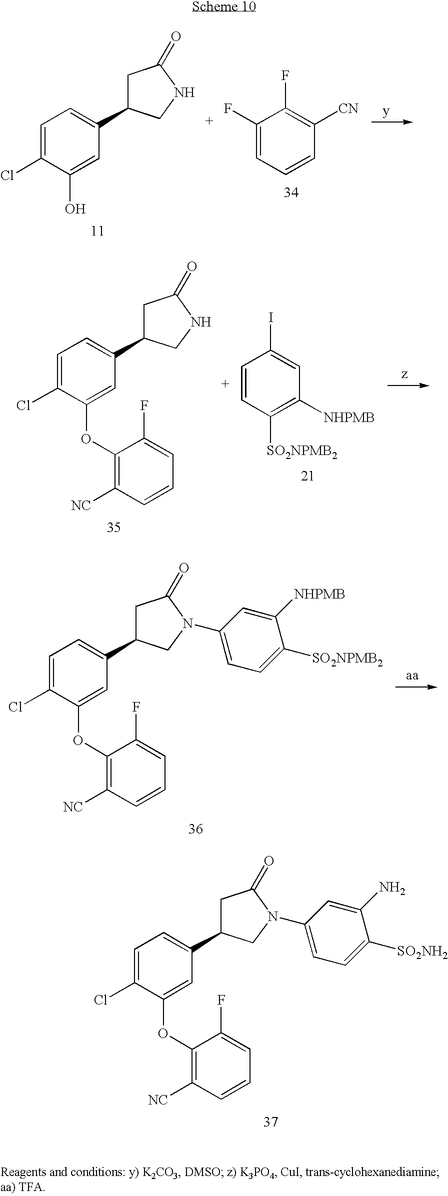

Scheme 10 illustrates another method of functionalizing the nitrogen of the lactam product from Scheme 3 (This is also applicable to the products of Scheme 1). First, the oxygen on the phenol moiety of compound II is functionalized by 34 in order to provide 35 (reaction y). Then protected bromo or iodo substituted compound 21 is added to 35 in order to produce compound 36 (reaction z). The protecting groups on compound 36 are subsequently removed to provide compound 37 (reaction aa).

The compounds of the present invention can be isolated and purified from the reaction mixture by purification strategies well known to those of skill in the art. For example, the compounds may be purified using known chromatographic procedures such as reverse phase HPLC, gel permeation, ion exchange, size exclusion, affinity, partition, or countercurrent distribution.

### V. Pharmaceutical Formulations

The compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical formulations comprising a pharmaceutically acceptable carrier or excipient and one or more compounds of the invention.

For preparing pharmaceutical formulations from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The dose administered to a patient, in the context of the present invention should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent of any adverse side-effects that accompany the administration of a particular compound in a particular patient.

The compound can also be introduced into an animal cell, preferably a mammalian cell, via a microparticles and liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidyl-ethanolamine (DOPE) is the basis of many “fusogenic” systems.

Such liposomes typically comprise a compound of choice and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91\17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments of the present invention, it is desirable to target the liposomes of the invention using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., J. Biol. Chem., 265:16337-16342 (1990) and Leonetti et al., PNAS 87:2448-2451 (1990).

In determining the effective amount of the compound to be administered in the treatment or prophylaxis of conditions owing to HIV infection, the physician evaluates circulating plasma levels of the compound, compound toxicities, progression of the disease, and the production of viral resistance to the compound.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted from 0.1 mg to 10 g, more typically 1.0 mg to 1 g, most typically 10 mg to 500 mg, according to the particular application and the potency of the active component. The composition can, if desired, also contain other compatible therapeutic or diagnostic agents. Administration can be accomplished via single or divided doses.

### VI. The Methods

The present invention also provides methods for treating or ameliorating HIV disease and related diseases. The method includes administering a therapeutically effective dosage of at least one compound of the invention to a subject suffering from HIV disease or HIV-related diseases. The invention also provides a method of combination therapy in which at least one compound of the invention is administered in combination with at least one other compound having activity against HIV disease or HIV-related disease.

### VI. a) Combination Therapies

In numerous embodiments, the compounds of the present invention will be administered in combination with one or more additional compounds or therapies. For example, multiple reverse transcriptase inhibitors can be co-administered, or one or more compound of the invention can be administered in conjunction with another therapeutic compound. In one embodiment, the other therapeutic agent is one that is used to prevent or treat HIV infection. In another embodiment, the other therapeutic agent is an agent used to treat an opportunistic infection associated with HIV infection and/or to treat or prevent HIV infection.

Suitable therapeutic agents for use in combination with the compounds of the present invention include, but are not limited to, protease inhibitors, non-nucleoside reverse transcriptase inhibitors, nucleoside reverse transcriptase inhibitors, antiretroviral nucleosides, entry inhibitors as well as other anti-viral agents effective to inhibit or treat HIV infection. Further examples of suitable therapeutic agents include, but are not limited to, zidovudine, didanosine, stavudine, interferon, lamivudine, adefovir, nevirapine, delaviridine, loviride, saquinavir, indinavir, and AZT. Other suitable therapeutic agents include, but are not limited to, antibiotics or other anti-viral agents, e.g., acyclovir.

Other combination therapies known to those of skill in the art can be used in conjunction with the compositions and methods of the present invention.

As explained above, it has now been discovered that pyrrolidones of the invention have anti-viral activity. As such, the compounds of the invention can be used to inhibit a wide variety of viruses and, thus, to treat a wide variety of viral infections in a human. Viruses that can be inhibited using the compounds of the invention include, but are not limited, to DNA viruses, RNA viruses as well as retroviruses. Examples of viruses that can be inhibited using the compounds include, but are not limited to, Herpes viruses, Hepatitis (A, B and C) viruses, influenza viruses, POX viruses, Rhino viruses and HTLV (Human T-cell Leukemia) viruses (e.g., HTLV 1 and 2). Based on their anti-viral activity, those of skill in the art will be aware of other viruses that can be treated using compounds of the invention.

### VI. b) Assays for Modulators of Reverse Transcriptase

Modulation of a reverse transcriptase, and corresponding modulation of HIV and viral infection, preferably inhibition, can be assessed using a variety of in vitro and in vivo assays, including cell-based models. Such assays can be used to test for inhibitors and activators of reverse transcriptase, and, consequently, inhibitors and activators of HIV infection and HIV-associated diseases. Such modulators of reverse transcriptase are useful for treating disorders related to HIV infection, as described herein. Modulators of reverse transcriptase are tested using either recombinant, chemically synthesized or naturally occurring reverse transcriptase.

Preferred modulators of the invention are those that act to decrease reverse transcriptase activity at the protein level. Preferred modulators also include those that decrease expression of reverse transcriptase at the nucleic acid level, e.g., inhibitors of the reverse transcriptase promoter, compounds that increase chromosome accessibility of the reverse transcriptase gene, compounds that decrease reverse transcriptase RNA stability and processing, and compounds that decrease reverse transcriptase RNA levels in the cytoplasm or nucleus.

Measurement of HIV infection modulation with a reverse transcriptase inhibitor, can be performed using a variety of assays, in vitro, in vivo, and ex vivo, as described herein. A suitable physical, chemical or phenotypic change that affects activity, e.g., enzymatic activity, cell proliferation (e.g., CD4+ lymphocyte proliferation), HIV replication, expression of HIV proteins, or ligand or substrate binding can be used to assess the influence of a test compound on the polypeptide of this invention. When the functional effects are determined using intact cells or animals, one can also measure a variety of effects, such as, viral RNA levels or viral titers in serum, ligand binding, transcriptional changes to both known and uncharacterized genetic markers (e.g., northern blots), changes in cell metabolism, changes related to cellular proliferation, viral marker expression, DNA synthesis, marker and dye dilution assays (e.g., GFP and cell tracker assays), etc.

### VI. c) In Vitro Assays

Assays to identify compounds with reverse transcriptase modulating activity can be performed in vitro. As described below, the assay can be either solid state or soluble. The protein may be bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the reverse transcriptase or a fragment thereof is contacted with a potential modulator and incubated for a suitable amount of time. In one embodiment, the potential modulator is bound to a solid support, and the reverse transcriptase is added. In another embodiment, the reverse transcriptase is bound to a solid support. A wide variety of assays can be used to identify reverse transcriptase-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator. Either the modulator or the known ligand or substrate is bound first, and then the competitor is added. After the reverse transcriptase is washed, interference with binding, either of the potential modulator or of the known ligand or substrate, is determined. Often, either the potential modulator or the known ligand or substrate is labeled.

### VI. d) Cell-Based In Vivo Assays

In another embodiment, reverse transcriptase is expressed in a cell, and functional, e.g., physical and chemical or phenotypic, changes are assayed to identify modulators of reverse transcriptase and modulators of HIV replication and HIV infected cells. Cells expressing reverse transcriptase can also be used in binding assays and enzymatic assays. Any suitable functional effect can be measured, as described herein. For example, cellular morphology (e.g., cell volume, nuclear volume, cell perimeter, and nuclear perimeter), ligand binding, lymphocyte proliferation, apoptosis, viral marker expression, GFP positivity and dye dilution assays (e.g., cell tracker assays with dyes that bind to cell membranes), DNA synthesis assays (e.g., ³H-thymidine and fluorescent DNA-binding dyes such as BrdU or Hoechst dye with FACS analysis), are all suitable assays to identify potential modulators using a cell based system. Suitable cells for such cell based assays include both primary cells such as PBMCs, lymphocytes (e.g., CD4+), neutrophils, polymorphonuclear leukocytes, and other phagocytic cells and cell lines, e.g., Jurkat cells, BJAB cells, etc. The reverse transcriptase can be naturally occurring or recombinant.

Cellular reverse transcriptase RNA and polypeptide levels can be determined by measuring the level of protein or mRNA. The level of reverse transcriptase or proteins related to reverse transcriptase are measured using immunoassays such as western blotting, ELISA and the like with an antibody that selectively binds to the reverse transcriptase polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, reverse transcriptase expression can be measured using a reporter gene system, e.g., utilizing a fusion protein or a gene linked to a reverse transcriptase promoter. Such a system can be devised using an reverse transcriptase protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

### VI. e) Animal Models

Animal models of HIV infection also find use in screening for modulators of reverse transcriptase. Similarly, transgenic animal technology including gene knockout technology, for example as a result of homologous recombination with an appropriate gene targeting vector, or gene overexpression, will result in the absence or increased expression of the reverse transcriptase. The same technology can also be applied to make knock-out cells. When desired, tissue-specific expression or knockout of the reverse transcriptase protein may be necessary.

Knock-out cells and transgenic mice can be made by insertion of a marker gene or other heterologous gene into an endogenous reverse transcriptase gene site in the mouse genome via homologous recombination. Such mice can also be made by substituting an endogenous reverse transcriptase with a mutated version of the reverse transcriptase gene by mutating an endogenous reverse transcriptase, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

### VI. f) Solid State and Soluble High Throughput Assays

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment the invention provides soluble assays using a reverse transcriptase protein, or a cell or tissue expressing an reverse transcriptase, either naturally occurring or recombinant. In another embodiment, the invention provides solid phase based in vitro assays in a high throughput format, where the reverse transcriptase is attached to a solid phase. Any one of the assays described herein can be adapted for high throughput screening.

In the high throughput assays of the invention, either soluble or solid state, it is possible to screen up to several thousand different modulators or ligands in a single day. This methodology can be used for reverse transcriptase in vitro, or for cell-based or membrane-based assays comprising an reverse transcriptase protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage. A tag for covalent or non-covalent binding can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as polyglycine sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

### EXAMPLES

The following examples are offered by way of illustration and not by way of limitation. Chemicals were purchased from Aldrich Chemical Co. (Milwaukee, Wis.), and used as received. Silica gel plates were obtained from Analtech (Newark, Del.). NMR spectra were recorded on a 300 MHz Brucker instrument. Chemical shifts are in ppm downfield from TMS and were recorded in the solvents listed. Splitting patterns are designated as follows: s, singlet; d, doublet; t, triplet; m, multiplet; br, broad.

### Example 1

### Preparation of 5

### 1.1 Synthesis of 2

4-chloro-3-hydroxy-benzaldehyde, 1, was prepared according to O. Langer, et al., Bioorg. Med. Chem., 9:677-694 (2001) and S. A. Adediran, et al., Bioorg. Med. Chem. 9:1175-1183 (2001). 26 mL of 64 mmol K₂CO₃ in DMF, and benzyl bromide (15.3 mmol) were added to the phenol, 1, (12.9 mmol), and the reaction was stirred at rt overnight. The next morning the reaction mixture was filtered and the filtrate was extracted into EtOAc, washed with saturated NH₄Cl, H₂O and brine, dried and concentrated. Purification with silica gel afforded the desired 3-benzyloxy-4-chloro-benzaldehyde compound, 2.

### 1.2 Results

Analytical data for structure 2 is provided below.

### 1.2a 3-Benzyloxy-4-chloro-benzaldehyde

¹H NMR (CDCl₃): 9.92 (s, 1H), 7.57 (d, 1H, J=8.0 Hz), 7.49 (d, 3H, J=7.2 Hz), 7.43-7.40 (m, 3H), 7.39-7.35 (m, 1H), 5.23 (s, 2H).

### 1.2 Synthesis of 3

A mixture of 3-benzyloxy-4-chloro-benzaldehyde compound, 2, (20 mmol), ammonium acetate (22 mL of 60 mmol in nitromethane) and 6 mL acetic acid was heated for 3 h. The reaction mixture was cooled down to rt and left overnight. A crystalline material formed, which was filtered and washed with a small amount of EtOAc and hexane to give a yellow product. The mother liquid was concentrated, dissolved in chloroform, washed with water and brine and dried. The material formed from the mother liquid was concentrated and purified by chromatography on silica gel (EtOAc:Hexane=1:3) to give more of the yellow product as a light yellow solid, 2-benzyloxy-1-chloro-4-(2-nitro-vinyl)-benzene, 3.

### 1.3 Results

Analytical data for structure 3 is provided below.

### 1.3a 2-Benzyloxy-1-chloro-4-(2-nitro-vinyl)-benzene

¹H NMR (CDCl₃): 8.06 (d, 1H, J=13.6 Hz), 7.66-7.48 (m, 7H), 7.26 (dd, 1H, J=2.0, 8.0 Hz), 7.21 (d, 1H, J=2.0 Hz), 5.35 (s, 2H). LCMS m/z 290.20 [M+H]⁺.

### 1.4 Synthesis of 4

50 mL of a solution of 3-acetyl-4-benzyl-oxazolidin-2-one (1 mmol) in anhydrous THF was added dropwise at −78° C. to lithium diisopropylamide in THF (1.0 eq) via syringe and stirred for 1 h. 25 mL of a solution of nitro olefin (1 mmol in anhydrous THF) was slowly added to the reaction mixture. The reaction was stirred for 1 h, quenched with saturated aqueous NH₄Cl and warmed to rt. The reaction mixture was extracted with EtOAc, dried, filtered, and concentrated. Chromatography of the residue on silica gel (EtOAc:Hexane) gave the desired product as white solid, 4-benzyl-3-[3-(3-benzyloxy-4-chloro-phenyl)-4-nitro-butyryl]-oxazolidin-2-one, 4.

### 1.5 Results

Analytical data for structure 4 is provided below.

### 1.5a 4-Benzyl-3-[3-(3-benzyloxy-4-chloro-phenyl)-4-nitro-butyryl]-oxazolidin-2-one

¹H NMR (CDCl₃): 7.48 (d, 2H, J=7.2 Hz), 7.40 (t, 2H, J=7.2 Hz), 7.36-7.28 (m, 5H), 7.17 (dd, 2H, J=1.6, 8.0), 6.90 (d, 1H, J=2.0 Hz), 6.83 (dd, 1H, J=2.0, 8.4 Hz), 5.16 (s, 2H), 4.69 (dd, 1H, J=6.8, 12.4 Hz), 4.62-4.54 (m, 2H), 4.18-4.07 (m, 3H), 3.51 (dd, 1H, J=7.6, 17.2 Hz), 3.30-3.21 (m, 2H), 2.74 (dd, 1H, J=9.6, 13.6 Hz); LCMS m/z 509.20 [M+H]⁺.

### 1.6 Synthesis of 5

4 was dissolved in EtOH (or partially dissolved). To this solution was added Raney Nickel. The mixture was placed under hydrogen and stirred for 10 to 24 h. The mixture was then filtered through celite and concentrated. The residue was purified on silica gel to give a pure product as a white solid, 4-(3-benzyloxy-4-chloro-phenyl)-pyrrolidin-2-one 5.

### 1.7 Results

Analytical data for structure 5 is provided below.

### 1.7a 4-(3-Benzyloxy-4-chloro-phenyl)-pyrrolidin-2-one

¹H NMR (CDCl₃): 7.41 (d, 2H, J=7.6 Hz), 7.35 (t, 2H, J=6.8 Hz), 7.29 (d, 2H, J=8.0 Hz), 6.78 (d, 1H, J=2.0 Hz), 6.75 (dd, 1H, J=2.0, 8.0 Hz), 5.58 (brs, 1H), 5.11 (s, 2H), 3.70 (t, 1H, J=8.4 Hz), 3.62-3.56 (m, 1H), 2.27 (dd, 1H, J=6.8, 9.2 Hz), 2.66 (dd, 1H, J=8.8, 16.8 Hz), 2.36 (dd, 1H, J=9.2, 17.2 Hz); LCMS m/z 302.20 [M+H]⁺.

### Example 2

### 2.1 Synthesis of 8

To a 750 mL solution of p-anisidine, 7, (18.48 g, 0.15 mol, 1 eq) and 2,5-dimethoxy-2,5-dihydrofuran, 6, (39.04 g, 0.3 mol, 2 eq) in acetonitrile, 600 mL of 0.4 N aqueous HCl solution was added. The reaction mixture was stirred at rt for 1 h, quenched with NaHCO₃ (40.32 g, 0.48 mol, 2 eq to HCl), concentrated under reduced pressure at 27° C. and partitioned between EtOAc and H₂O. The aqueous phase was extracted with EtOAc and the combined organic extracts were washed with brine, dried over Na₂SO₄ and concentrated. The crude residue was purified by chromatography on silica column with a mixed solvent of hexanes:EtOAc (1:1) to give 1-(4-methoxy-phenyl)-1,5-dihydro-pyrrol-2-one, 8 (10.85 g, 38%).

### 2.2 Results

Analytical data for structure 8 is provided below.

### 2.2a 1-(4-Methoxy-phenyl)-1,5-dihydro-pyrrol-2-one

¹H NMR (CDCl₃): δ 7.57 (2H, d, J=9.2 Hz), 7.14 (1H, dt, J=0.8, 6.0 Hz), 6.92 (2H, d, J=9.2 Hz), 6.26 (1H, dt, J=0.8, 6.0 Hz), 4.40 (2H, t, J=1.6), 3.80 (3H, s).

### 2.3 Synthesis of 9

A mixture of 2-benzyloxy-4-bromo-1-chloro-benzene (120 g, 0.4 mol), diborate (107.5 g, 0.42 mol), KOAc (117.8 g, 3 eq), Pd(dppf)₂Cl₂ (1% mole) and 500 mL DMF was degassed, with stirring, and recharged with nitrogen. The mixture was heated to 80° C. for 3 h. DMF was removed under vacuum. The residue was mixed with ethyl acetate. After filtration, the solid was washed with ethyl acetate (3×20 mL) and re-crystallized in ethyl acetate to give 97 g pure product. Mother solution was also concentrated. The residue was purified by washing short silica column (10% ethyl acetate:hexane) to give crude product, which was re-crystallized in ethyl acetate to provide 2-(4-chloro-3-benzyloxy-phenyl)-4,4,5,5,-tetramethyl[1,3,2]dioxaborolane, 9.

### 2.4 Results

Analytical data for structure 9 is provided below.

### 2.4a 2-(4-Chloro-3-benzyloxy-phenyl)-4,4,5,5,-tetramethyl[1,3,2]dioxaborolane

¹H NMR (CDCl₃): δ7.35 (2H, d, J=7.6 Hz), 7.28 (1H, br s), 7.17-7.26 (5H, m), 7.11 (1H, s), 5.02 (2H, s), 1.20 (12H, s).

### 2.5 Synthesis of 10

A solution of 1-(4-methoxy-phenyl)-1,5-dihydro-pyrrol-2-one, 8, (9 g, 47.57 mmol, 1 eq), 2-(3-benzyloxy-4-chloro-phenyl)-4,4,5,5-tetramethyl-[1,3,2]dioxaborolane, 9, (32.8 g, 95.14 mmol, 2 eq), chloro(1,5-cyclooctadiene)rhodium (I) dimer (352 mg, 0.7136 mmol, 0.015 eq), (R)-BINAP (1.04 g, 1.665 mmol, 0.035 eq) and K₂CO₃ (3.3 g, 23.8 mmol, 0.5 eq) was prepared in a mixed solvent of dioxane (200 mL) and water (20 mL). The solution was purged with nitrogen and heated to 80° C. in an oil bath for 26 h. The reaction mixture was partitioned between EtOAc and brine. The aqueous phase was extracted with EtOAc and the combined EtOAc extracts were washed with brine, dried over Na₂SO₄, and evaporated. Chromatography through a short column with eluent of 5:4 hexanes:EtOAc gives a solid product that was recrystallized in EtOAc and hexanes to provide (R)-4-(3-Benzyloxy-4-chloro-phenyl)-1-(4-methoxy-phenyl)-pyrrolidin-2-one, 10.

### 2.6 Results

Analytical data for structure 10 is provided below.

### 2.6a (R)-4-(3-Benzyloxy-4-chloro-phenyl)-1-(4-methoxy-phenyl)-pyrrolidin-2-one

¹H NMR (CDCl₃): δ 7.48 (2H, d, J=9.2 Hz), 7.44 (2H, m), 7.32-7.39 (4H, m), 6.92 (2H, d, J=9.2 Hz), 6.86 (1H, d, J=1.6 Hz), 6.83 (1H, dd, J=0.8, 8.0 Hz), 5.16 (2H, s), 4.12 (1H, dd, J=8.0, 9.6 Hz), 3.81 (3H, s), 3.75 (1H, dd, J=6.8, 9.6 Hz), 3.63 (1H, dddd, J=8 Hz), 2.98 (1H, dd, J=8.8, 16.8 Hz), 2.68 (1H, dd, J=16.8, 8.4 Hz) ppm.

### 2.7 Synthesis of 5

To a 1400 mL solution of 4-(3-benzyloxy-4-chloro-phenyl)-1-(4-methoxy-phenyl)-pyrrolidin-2-one, 10, (16.5 g, 40 mmol, 1 eq) in CH₃CN, a solution of ammonium cerium nitrate (CAN) (65.8 g, 0.12 mol, 3 eq) in 50% aqueous CH₃CN was added dropwise at 0° C. The reaction mixture was stirred at 0° C. for 1 h and Na₂SO₃ (45.4 g, 0.36 mol, 9 eq) was added. The reaction mixture was stirred at 0° C. for 1 h and filtered through Celite to remove the precipitate. The mother liquor was concentrated under reduced pressure and the residue was partitioned between 5% MeOH in EtOAc and H₂O. The aqueous phase was extracted with 5% MeOH in EtOAc and the combined extracts were washed with brine, dried over Na₂SO₄, and evaporated. Chromatography through a short column with eluent of 40:1 CH₂Cl₂:MeOH afforded (R)-4-(3-benzyloxy-4-chloro-phenyl)-pyrrolidin-2-one, 5.

### Example 3

### Preparation of 12

### 3.1 Synthesis of 11

To a mixture of 5 (10.5 g, 34.8 mmol) and 3.2 g of Pd/C (10%), 500 mL THF, and 500 μL of 4M HCl in dioxane was added. The reaction mixture was degassed and purged with hydrogen 10 times, stirred under atmospheric hydrogen for 19 h, and filtered through Celite. Evaporation of the filtrate afforded crude product that was recrystallized in EtOAc and hexanes to provide (R)-4-(4-chloro-3-hydroxy-phenyl)-pyrrolidin-2-one, 11.

### 3.2 Synthesis of 12

To a solution of 11 (3.57 g, 16.9 mmol, 1 eq) and 3-chloro-2-fluorobenzonitrile (5.25 g, 33.74 mol, 2 eq) in dry DMSO (125 mL) was added K₂CO₃ (7 g, 50.6 mmol, 3.1 eq). The reaction mixture was stirred at rt for 24 h and partitioned between EtOAc and brine. The aqueous phase was extracted with EtOAc and the combined EtOAc extracts were washed with brine, dried over Na₂SO₄, and evaporated. Chromatography on a silica column with eluent of 20:1 CH₂Cl₂:MeOH afforded (R)-3-chloro-2-[2-chloro-5-(5-oxo-pyrrolidin-3-yl)-phenoxy]-benzonitrile, 12.

### 2.4 Results

Analytical data for structure 9 is provided below.

### 2.4a (R)-3-chloro-2-[2-chloro-5-(5-oxo-pyrrolidin-3-yl)-phenoxy]-benzonitrile

MS m/z 347.0 (M+1).

### Example 4

### Preparation of 26

### 4.1 Synthesis of 22

To a dry round bottom flask was placed 0.5 mmol of 12, 0.9 mmol potassium phosphate, 2-fluoro-5-iodobenzonitrile (0.75 mmol), 1,2-trans-cyclohexanediamine (60 μL) and CuI (80 mg) under nitrogen. The reaction was charged with nitrogen, DMF (5 mL) and dioxane (5 mL). The mixture was stirred and heated at 110° C. for 20 h and then cooled to rt. The rt solution was diluted with EtOAc and filtered. The filtrate was washed with saturated ammonium chloride, water and brine and dried. Concentration and chromatography of the residue on silica gel gives pure 3-chloro-2-{2-chloro-5-[1-(3-cyano-4-fluoro-phenyl)-5-oxo-pyrrolidin-3-yl]-phenoxy}-benzonitrile, 22.

### 4.2 Results

Analytical data for structure 22 is provided below.

### 4.2a 3-chloro-2-{2-chloro-5-[1-(3-cyano-4-fluoro-phenyl)-5-oxo-pyrrolidin-3-yl]-phenoxy}-benzonitrile

MS m/z 466 (M+1).

### 4.3 Synthesis of 23

To a solution of 22 (20 mg, 0.043 mmol) in propanol (0.5 mL) was added hydrazine (17 mg, 0.344 mmol). The mixture was stirred at 100° C. for 14 h and then cooled to rt. Solvent was removed under vacuum and the residue was purified by reverse phase LC/MS to provide 2-{5-[1-(3-amino-1H-indazol-5-yl)-5-oxo-pyrrolidin-3-yl]-2-chloro-phenoxy}-3-chloro-benzonitrile, 23.

### 4.4 Results

Analytical data for structure 23 is provided below.

### 4.4a 2-{5-[1-(3-Amino-1H-indazol-5-yl)-5-oxo-pyrrolidin-3-yl]-2-chloro-phenoxy}-3-chloro-benzonitrile

¹H NMR 400 MHz (MeOD): δ 7.99 (1H, d), 7.94 (1H, dd), 7.85 (1H, dd), 7.78 (1H, dd), 7.56 (1H, d), 7.47 (1H, d), 7.43 (1H, t), 7.21 (1H, d), 6.64 (1H, d), 4.28 (1H, dd), 3.89 (1H, dd), 3.74 (1H, m), 3.01 (1H, dd), 2.67 (1H, dd); MS m/z 478 (M+1).

### 4.5 Synthesis of 25

2-{5-[1-(3-Amino-1H-indazol-5-yl)-5-oxo-pyrrolidin-3-yl]-2-chloro-phenoxy}-3-chloro-benzonitrile, 23 (80 mg, 0.167 mmol) and phthalic anhydride (30 mg, 0.2 mmol) was added into 1 mL dioxane and heated to 120° C. After 3 h, the mixture was cooled down to rt and the solvent was removed under vacuum. The residue was purified by flash chromatography (silica gel, 0-8% MeOH in CH₂Cl₂) to provide 3-chloro-2-(2-chloro-5-{1-[3-(1,3-dioxo-1,3-dihydro-isoindol-2-yl)-1H-indazol-5-yl]-5-oxo-pyrrolidin-3-yl}-phenoxy)-benzonitrile, 25, as a light yellow solid.

### 4.6 Synthesis of 26

25 (62 mg, 0.102 mmol) in anhydrous CH₂Cl (2 mL) was treated with chlorosulfonyl isocyanide (20 μL). After stirring at rt for 1 h, solvent was removed. The residue was treated with water (1 mL) and stirred at rt for 1 h. Water was then removed under vacuum, leaving a residue. The residue was dissolved in EtOH (2 mL) and treated with hydrazine (60 μL) for 3 h. Solvent was then removed under vacuum and the residue was purified by flash chromatography (silica gel, 0-5% MeOH in CH₂Cl₂) to provide 3-amino-5-{4-[4-chloro-3-(2-chloro-6-cyano-phenoxy)-phenyl]-2-oxo-pyrrolidin-1-yl}-indazole-1-carboxylic acid amide, 26, as a white solid.

### 4.7 Results

Analytical data for structure 26 is provided below.

### 4.7a 3-Amino-5-{4-[4-chloro-3-(2-chloro-6-cyano-phenoxy)-phenyl]-2-oxo-pyrrolidin-1-yl}-indazole-1-carboxylic acid amide

¹H NMR 400 MHz (MeOD): δ 8.10 (1H, d), 7.83 (1H, d), 7.80 (1H, dd), 7.73 (1H, dd), 7.67 (1H, dd), 7.53 (1H, d), 7.37 (1H, t), 7.17 (1H, d), 6.58 (1H, d), 4.26 (1H, dd), 3.86 (1H, dd), 3.71 (1H, m), 3.00 (1H, dd), 2.61 (1H, dd); MS m/z 521 (M+1).

### Example 5

### Preparation of 29

### 5.1 Synthesis of 28

12 (200.0 mg, 0.58 mmol) was coupled with 4-bromo-2-nitrobenzonitrile, 27, (156.9 mg, 0.69 mmol) to provide (R)-3-chloro-2-(2-chloro-5-(1-(4-cyano-3-nitrophenyl)-5-oxopyrrolidin-3-yl)phenoxy)benzonitrile, 28, as a light yellow solid.

### 5.2 Results

Analytical data for structure 28 is provided below.

### 5.2a (R)-3-chloro-2-(2-chloro-5-(1-(4-cyano-3-nitrophenyl)-5-oxopyrrolidin-3-yl)phenoxy)benzonitrile

¹H NMR 400 MHz (MeOD): δ 8.88 (1H, d), 7.99 (2H, m), 7.85 (1H, dd), 7.78 (1H, dd), 7.54 (1H, d), 7.44 (1H, t), 7.19 (1H, dd), 6.64 (1H, d), 4.30 (1H, dd), 3.88 (1H, dd), 3.72 (1H, m), 3.00 (1H, dd), 2.73 (1H, dd); MS m/z 493.0 (M+1).

### 5.3 Synthesis of 29

A mixture of 28 (277.7 mg, 0.56 mmol) and SnCl₂.2H₂O (393.8 mg, 1.75 mmol) in 5.6 mL EtOH was heated at 50° C. under N₂ atmosphere for 2 h. The reaction mixture was then diluted with 6 mL water and made alkaline with 1N NaOH aqueous solution until the pH was between 9 and 10. 6 mL EtOAc was then added. The mixture was stirred at rt for 1 h before filtering through celite. The two layers of the permeate were separated, and the aqueous layer was extracted with EtOAc. The combined EtOAc layer was washed with brine, dried over MgSO₄, concentrated and purified by silica gel chromatography (hexanes:EtOAc) to give (R)-2-amino-4-(4-(4-chloro-3-(2-chloro-6-cyanophenoxy)phenyl)-2-oxopyrrolidin-1-yl)-benzamide, 29, as a yellow solid.

### 5.4 Results

Analytical data for structure 29 is provided below.

### 5.4a (R)-2-amino-4-(4-(4-chloro-3-(2-chloro-6-cyanophenoxy)phenyl)-2-oxopyrrolidin-1-yl)-benzamide

¹H NMR 400 MHz (MeOD): δ 7.81 (1H, dd), 7.74 (1H, dd), 7.52 (2H, m), 7.39 (1H, t), 7.13 (1H, dd), 6.94 (1H, d), 6.83 (1H, dd), 6.51 (1H, d), 4.18 (1H, dd), 3.76 (1H, dd), 3.64 (1H, m), 2.97 (1H, dd), 2.55 (1H, dd); MS m/z 481.0 (M+1).

### Example 6

### Preparation of 30

### 6.1 Synthesis of 30

25 (10 mg, 0.016 mmol) was dissolved in 0.5 mL pyridine and 10 μL methanesulfonyl chloride was added. The mixture was stirred at rt for 16 h. Solvent was removed under vacuum and the residue was treated with saturated ammonium chloride (2 mL) followed by extraction (3 mLx3). The extracts were combined, concentrated and redissolved in 0.5 mL EtOH. 5 μL Hydrazine was added, and the mixture was stirred at rt for 3 h. Solvent was removed under vacuum and the residue was purified by reverse phase LC/MS to provide 2-{5-[1-(3-amino-1-methanesulfonyl-1H-indazol-5-yl)-5-oxo-pyrrolidin-3-yl]-2-chloro-phenoxy}-3-chloro-benzonitrile, 30.

### 6.2 Results

Analytical data for structure 30 is provided below.

### 6.2a 2-{5-[1-(3-Amino-1-methanesulfonyl-1H-indazol-5-yl)-5-oxo-pyrrolidin-3-yl]-2-chloro-phenoxy}-3-chloro-benzonitrile

¹H NMR 400 MHz (MeOD): δ 7.94 (1H, d), 7.81-7.95 (3H, m), 7.75 (1H, dd), 7.54 (1H, d), 7.39 (1H, t), 7.18 (1H, dd), 6.60 (1H, d), 4.28 (1H, dd), 3.88 (1H, dd), 3.72 (1H, m), 2.93 (3H, s), 3.00 (1H, dd), 2.64 (1H, dd); MS m/z 556 (M+1).

### Example 7

### Preparation of 31

### 7.1 Synthesis of 31

Following the same procedure for the preparation of compound 30 in example 6 and 7, the titled compound was made.

### 7.2 Results

Analytical data for structure 31 is provided below.

### 7.2a 2-{5-[1-(3-amino-1-methanesulfonyl-1H-indazol-5-yl)-5-oxo-pyrrolidin-3-yl]-2-chloro-phenoxy}-3-fluoro-benzonitrile

¹H NMR 400 MHz (DMSO-d6): δ 7.85 (1H, d, J=2.0), 7.81 (1H, dd, J=2.4, 5.2 Hz), 7.65-7.69 (2H, m), 7.58 (1H, d, J=9.2 Hz), 7.46 (1H, d, J=8.0 Hz), 7.34 (1H, dt, J=4.8, 8.0 Hz), 7.14 (1H, dd, J=2.0, 8.4 Hz), 6.94 (1H, s), 6.40 (2H, s), 3.99 (1H, t, J=8.4 Hz), 3.66 (1H, t, J=8.0 Hz), 3.59 (1H, ddd, J=8.4 Hz), 3.15 (3H, s), 2.70 (1H, dd, J=8.4, 16.4 Hz), 2.55 (1H, dd, J=8.4, 16.4 Hz).

### Example 8

### Preparation of 17

### 8.1 Synthesis of 15

A stirred solution of N-(4-methoxy-benzyl)-methanesulfonamide, 14, (613 mg, 2.85 mmol) in anhydrous DMF was cooled to 0° C. with an ice-water bath and treated with a 60% dispersion of sodium hydride (114 mg, 4.75 mmol). A 5 mL solution of 2-fluoro-5-iodobenzonitrile, 13, in anhydrous DMF was added to the reaction mixture. The resulting solution was allowed to warm to rt and was heated at 80° C. for 1 h. The reaction was cooled to rt and quenched with saturated aqueous NH₄Cl, extracted with EtOAc, dried with Na₂SO₄, filtered, and concentrated. Chromatography (SiO₂, 3:1 Hex:EtOAc) gives N-(2-cyano-4-iodo-phenyl)-N-(4-methoxy-benzyl)-methane-sulfonamide, 15, as a white solid.

### 8.2 Synthesis of 16

6 mL of a solution of 15 (600 mg, 1.4 mmol) in 3:2:1 THF:MeOH:H₂O was treated with sodium hydroxide (1.0 g, 27.0 mmol). The reaction mixture was heated under reflux for 12 h. The reaction mixture was allowed to cool to rt and washed with ethanol. The aqueous layer was acidified to pH 1 and extracted with EtOAc. The organic layer was dried with Na₂SO₄, filtered and concentrated to give 5-iodo-2-[methanesulfonyl-(4-methoxy-benzyl)-amino]-benzoic acid, 16, as a white solid.

### 8.3 Results

Analytical data for structure 16 is provided below.

### 8.3a 5-iodo-2-[methanesulfonyl-(4-methoxybenzyl)-amino]-benzoic acid

¹H NMR (CDCl₃): δ 8.34 (1H, d, J=2.0 Hz), 7.76 (1H, dd, J=2.0, 8.0 Hz), 7.15 (2H, d, J=8.8 Hz), 6.79 (3H, m), 4.51 (1H, br s), 3.78 (3H, s), 2.98 (3H, s).

### 8.4 Synthesis of 17

A solution of 16 (366 mg, 0.79 mmol) in CH₂Cl₂ (10 mL) was stirred at 0° C. and treated with methanesulfonyl chloride (183 mg, 1.6 mmol) and Et₃N (162 mg, 1.6 mmol). The reaction was stirred for 1 h and treated with bis-(4-methoxy-benzyl)-amine (257 mg, 0.87 mmol). The reaction was allowed to warm to rt and washed with saturated aqueous NaHCO₃, dried with Na₂SO₄, and concentrated. Chromatography (SiO₂, 1:1 Hex:EtOAc) gives 5-iodo-2-[methanesulfonyl-(4-methoxy-benzyl)-amino]-N,N-bis-(4-methoxy-benzyl)-benzamide, 17, as a white solid.

### 8.5 Results

Analytical data for structure 17 is provided below.

### 8.5a 5-Iodo-2-[methanesulfonyl-(4-methoxy-benzyl)-amino]-N,N-bis-(4-methoxy-benzyl)-benzamide

¹H NMR (CDCl₃): δ 7.63 (1H, d, J=2.0 Hz), 7.56 (1H, dd, J=2.0, 8.4 Hz), 7.30 (2H, d, J=8.4 Hz), 7.18-7.21 (2H, m), 7.08 (2H, d, J=8.4 Hz), 6.91 (2H, d, J=8.4 Hz), 6.88 (2H, J=8.4 Hz), 6.38-6.55 (2H, m), 6.67-6.70 (1H, m), 5.36 (1H, m), 4.71 (2H, m), 4.42 (1H, m), 4.12 (1H, m), 3.96 (1H, m), 3.83 (3H, s), 3.81 (3H, s), 3.79 (3H, s).

### Example 9

### Preparation of 33

### 9.1 Synthesis of 32

To a dry round bottom flask was placed 0.5 mmol of 12, 0.9 mmol potassium phosphate, 17 (0.75 mmol), 1,2-trans-cyclohexanediamine (60 μL) and CuI (80 mg) under nitrogen. The reaction was charged with nitrogen, DMF (5 mL) and dioxane (5 mL). The mixture was stirred and heated at 110° C. for 20 h and then cooled to rt. The rt solution was diluted with EtOAc and filtered. The filtrate was washed with saturated ammonium chloride, water and brine and dried. Concentration and chromatography of the residue on silica gel gives pure 32.

### 9.2 Synthesis of 33

32 (200 mg) was dissolved in 3 mL of TFA and the resulting solution was heated at 80° C. for 4 h, and then cooled to rt. TFA was removed under reduced pressure and the residue was purified by silica gel column chromatography (EtOAc:MeOH=20:1) to give pure 5-{4-[4-chloro-3-(2-chloro-6-cyano-phenoxy)-phenyl]-2-oxo-pyrrolidin-1-yl}-2-methanesulfonylamino-benzamide, 33.

### 9.3 Results

Analytical data for structure 33 is provided below.

### 9.3a 5-{4-[4-chloro-3-(2-chloro-6-cyano-phenoxy)-phenyl]-2-oxo-pyrrolidin-1-yl}-2-methanesulfonylamino-benzamide

¹H NMR 400 MHz (CDCl₃): δ 10.9 (1H, br s), 7.88 (1H, dd), 7.86 (1H, br s), 7.81-7.74 (3H, m), 7.53 (1H, d), 7.46 (1H, d), 7.41 (1H, apparent t), 7.15 (1H, d), 7.06 (1H, br s), 6.75 (1H, s), 4.09 (1H, d), 3.74 (1H, dd), 3.61 (1H, m), 2.92 (3H, s), 2.73 (1H, dd), 2.48 (1H, dd); MS m/z 559.0 (M+1).

### Example 10

### Preparation of 21

### 10.1 Synthesis of 19

To a solution of p-anisaldehyde (146 mmol) in ethanol (500 mL) was added 4-methoxybenzylamine (146 mmol). The mixture was then cooled in an ice water bath. NaBH₄ (294 mmol) was added in portions and the reaction mixture was allowed to warm to rt, gradually. Ice water slush (100 mL) was added and the mixture was concentrated to half its original volume. The mixture was then extracted with ether and the organics were combined, washed with brine, dried, and concentrated. Hexane (˜50 mL) was added and the precipitate was filtered to give 35 g of bis-(4-methoxy-benzyl)-amine, 19, as white solid.

### 10.2 Synthesis of 20

To a solution of 19 (9.41 g), 10.2 mL of triethylamine and 100 mL dichloromethane was added. 10 g of 2-fluoro-4-bromobenzenesulfonyl chloride was then added in portions at 0° C. with stirring. The reaction mixture was warmed to rt slowly and stirred overnight. Dichloromethane (100 mL) was added and the mixture was washed with 1N HCl solution, saturated NaHCO₃, brine and dried. After removal of the solvent, the solid was mixed with hexane and filtered to give pure 4-bromo-2-fluoro-N,N-bis-(4-methoxy-benzyl)-benzenesulfonamide, 20. The mother solution was concentrated and purified to give more product.

### 10.3 Results

Analytical data for structure 20 is provided below.

### 10.3a 4-Bromo-2-fluoro-N,N-bis-(4-methoxy-benzyl)-benzenesulfonamide

¹H NMR (CDCl₃): δ 7.74 (1H, t, J=7.6 Hz), 7.36 (2H, dt, J=8.4, 1.6 Hz), 6.98 (4H, d, J=8.4 Hz), 6.76 (4H, d, J=8.4 Hz), 4.33 (4H, s), 3.78 (6H, s).

### 10.4 Synthesis of 21

To a 20 mL microwave tube was added 760 mg K₂CO₃, 20 (2 g), 660 uL 4-methoxybenzylamine, and 18 mL DMF. The tube was heated in a microwave reactor at 180° C. for 2500 seconds and cooled. This reaction was performed 10 times so that a total of 20 g of 2-fluoro-sulfonamide was used. The organic from the 10 reactions was combined, extracted with ethyl acetate, washed with saturated ammonium chloride solution and water. It was dried, concentrated and the residue was re-crystallized from hexane ethyl acetate to give the desired 4-bromo-N,N-bis-(4-methoxybenzyl)-2-(4-methoxy-benzylamino)-benzenesulfonamide, 21.

### 10.4 Results

Analytical data for structure 21 is provided below.

### 10.4a 4-Bromo-N,N-bis-(4-methoxy-benzyl)-2-(4-methoxy-benzylamino)-benzenesulfonamide

¹H NMR (CDCl₃): δ 7.60 (1H, d, J=8.4 Hz), 7.15 (2H, d, J=8.4 Hz), 6.94 (4H, d, J=8.4 Hz), 6.88 (1H, d, J=1.6 Hz), 6.80-6.85 (3H, m), 6.77 (4H, d, J=8.4 Hz), 6.35 (1H, t, J=4.8 Hz), 4.24 (2H, d, J=4.8 Hz), 4.18 (4H, s), 3.78 (9H, s).

### Example 11

### Preparation of 37

### 11.1 Synthesis of 35

To a solution of 11 (3.57 g, 16.9 mmol, 1 eq) and 3-fluoro-2-fluorobenzonitrile (5.25 g, 33.74 mol, 2 eq) in dry DMSO (125 mL) was added K₂CO₃ (7 g, 50.6 mmol, 3.1 eq). The reaction mixture was stirred at rt for 24 h and partitioned between EtOAc and brine. The aqueous phase was extracted with EtOAc and the combined EtOAc extracts were washed with brine, dried over Na₂SO₄, and evaporated. Chromatography on a silica column with eluent of 20:1 CH₂Cl₂:MeOH afforded (R)-3-chloro-2-[2-chloro-5-(5-oxo-pyrrolidin-3-yl)-phenoxy]-benzonitrile, 35.

### 11.2 Results

Analytical data for structure 35 is provided below.

### 11.2a 5-{4-[4-chloro-3-(2-chloro-6-cyano-phenoxy)-phenyl]-2-oxo-pyrrolidin-1-yl}-2-methanesulfonylamino-benzamide

¹H NMR (CDCl₃): δ7.51 (1H, dt, J=1.2, 7.6 Hz), 7.43 (1H, d, J=8.0 Hz), 7.42 (1H, dt, J=1.6, 10.4 Hz), 7.31 (1H, dd, J=4.8, 8.0 Hz), 7.00 (1H, dd, J=2.0, 8.0 Hz), 6.65 (1H, brs), 5.61 (1H, br s), 3.73 (1H, t, J=9.2 Hz), 3.60 (1H, dddd, J=8.4 Hz), 3.31 (1H, dd, J=6.8, 9.2 Hz), 2.68 (1H, dd, J=9.2, 17.2 Hz), 2.37 (1H, dd, J=8.4, 16.8 Hz).

### 11.3 Synthesis of 36

To a dry round bottom flask is placed 35 (0.5 mmole), potassium phosphate (0.9 mmol), 21 (0.75 mmol), 1,2-trans-cyclohexanediamine (60 μL) and CuI (80 mg) under nitrogen. The reaction is charged with nitrogen, DMF (5 mL) and dioxane (5 mL). The mixture is stirred and heated at 110° C. for 20 h and then cooled to rt. It is diluted with EtOAc and filtered. The filtrate is washed with saturated ammonium chloride, water and brine, and then dried. Concentration and chromatography of the residue on silica gel gave 4-{4-[4-chloro-3-(2-cyano-6-fluoro-phenoxy)-phenyl]-2-oxo-pyrrolidin-1-yl}-N,N-bis-(4-methoxy-benzyl)-2-(4-methoxy-benzylamino)-benzenesulfonamide, 36.

### 11.4 Synthesis of 37

36 (120 mg) was dissolved in 2 mL of 50:50 TFA:dichloromethane. The solution was stirred at rt overnight and concentrated. The residue was dissolved in chloroform and treated with saturated sodium bicarbonate solution. The resulting mixture was stirred for 10 min; the organic layer was then separated, washed with brine and dried over sodium sulfate. After removal of the solvent, the residue was purified on silica gel (EtOAc:MeOH=50:1) to give pure 2-amino-4-{4-[4-chloro-3-(2-cyano-6-fluoro-phenoxy)-phenyl]-2-oxo-pyrrolidin-1-yl}-benzenesulfonamide, 37.

### 11.5 Results

Analytical results for structure 37 are provided below.

### 11.5a 2-amino-4-{4-[4-chloro-3-(2-cyano-6-fluoro-phenoxy)-phenyl]-2-oxo-pyrrolidin-1-yl}-benzenesulfonamide

¹H NMR (CDCl₃): δ 7.55-7.60 (2H, m), 7.48 (1H, d, J=6.4 Hz), 7.46 (1H, d, J=6.4 Hz), 7.40 (1H, dt, J=4.8, 8.0 Hz), 7.17 (2H, m), 6.95 (1H, s), 6.83 (1H, dd, J=2.4, 9.2 Hz), 6.34 (2H, br s), 5.52 (2H, br s), 4.08 (1H, m), 3.59-3.70 (2H, m), 2.76 (1H, dd, J=8.4, 16.4 Hz), 2.51 (1H, dd, J=8.4, 16.4).

By repeating the procedures described in the above examples, using appropriate starting materials, the following compounds of the invention, as identified in the Table 1, were obtained.

### Example 12

### Biological Testing Assay

The ability of the compounds of the invention to inhibit HIV was tested by the following assay. Vesicular stomatitis virus-glycoprotein (“VSV-g”) pseudotyped HIV-1 luciferase reporter virus (“HIV-1 pseudovirions”) was used in this assay. The virus was harvested from Human Embryonic Kidney (HEK) 293T producer cells (“HEK293T”) following a triple transient transfection (CaP, Clontech) of the three plasmid HIV-1 lentiviral vector system comprised of the VSV-g envelope expression plasmid, packaging construct (delta psi) and the HIV-1 LTR:Luc plasmid. The VSV-g envelope expression plasmid generates the pseudovirus receptor that permits a broad tropism and mediates entry into the HEK293T target cells. The delta psi packaging construct supplies all of the structural and regulatory gene products needed to generate the pseudovirus. The viral vector RNA synthesized from the HIV-1 LTR:Luc plasmid possesses the cis RNA packaging signal (psi sequence) in addition to the luciferase reporter gene and the HIV-1 LTR. The supernatants of transfected producer cells contain HIV-1 pseudovirions carrying only the luciferase gene in the viral genome. Upon transduction of the target 293T cells, the viral genomic RNA will undergo reverse transcription, nuclear translocation, integration, and transcription of the integrated luciferase gene driven by the PGK (phosphoglycerate kinase promoter). Luciferase activity using Bright-Glo reagent (Promega) substrate was measured 48 hr post-infection using a CLIPR plate reader (Molecular Devices) to determine EC50 values.

### Biological Testing Assay Protocol

### Activity Against Replication-Defective HIV Reporter Virus (Single-Cycle Infection Assay) in 293T Target Cells:

HEK 293T cells are routinely cultured in Dulbecco's Modified Eagle Medium (DMEM) supplemented with 10% FBS, 1× Pen/Strep/Glutamine. The protocol is as follows:
1. 293T cells are seeded in the 1536-well format at 700 cells/well (5 uL volume) using an Aquamax (Molecular Devices) liquid dispenser.
2. Cells are cultured at 37° C. under 5% CO₂ for 24 hours.
3. 50 mL of each compound (serially diluted in DMSO) are transferred using the PinTool (GNF).
4. After a 1 hr, 37° C. incubation, HIV reporter virus is transferred to the cells using the Aquamax in a volume of 2 μL corresponding to a multiplicity of infection (MOI) of approximately 1.0.
5. The treated and infected cells are cultured for an additional 48 hr at 37° C.
6. Luciferase activity is monitored by addition of Bright-Glo (Promega, Cat.# E263B and E264B) luciferase reagent (5 uL/well, Aquamax) followed by plate reading on the CLIPR apparatus (Molecular Devices) using a 20 second shuttle speed.
   Cytotoxicity Assay (in Parallel with all Infection Inhibition Assays)
1. 293T cells are seeded in the 1536-well format at 700 cells/well (5 μL volume) using an Aquamax (Molecular Devices) liquid dispenser.
2. Cells are cultured at 37° C. under 5% CO₂ for 24 hours.
3. 50 mL of each compound (serially diluted in DMSO) are transferred using the PinTool (GNF).
4. The treated and uninfected cells are cultured for an additional 48 hr at 37° C.
5. Cell viability is assessed by addition of 1 uL of Alamar Blue (Promega, Cat.# 00-100) diluted 1:1 in DMEM.
6. Cells are further incubated for 4 hr at room temperature and subsequent fluorescence intensity is read using an Acquest (TREK systems) with a 50/50 beam splitter.

It was understood that the examples and embodiments described herein were for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the formula: 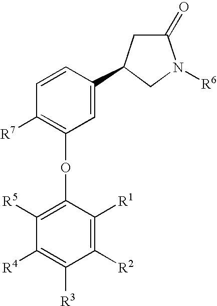
wherein R¹ and R⁵ are members independently selected from H, CN, halogen, substituted or unsubstituted C₁-C₄ alkyl, substituted or unsubstituted C₂-C₄ alkenyl and OR⁸;
wherein R⁸ is a member selected from substituted or unsubstituted C₁-C₄ alkyl and C₁-C₄ haloalkyl;
R² and R⁴ are members selected from H, halogen, CN, and substituted or unsubstituted C₁-C₄ alkyl;
R³ is a member selected from H, CN, and alkyl;
R⁶ is a member selected from a fused phenyl heterocyclic ring system and: 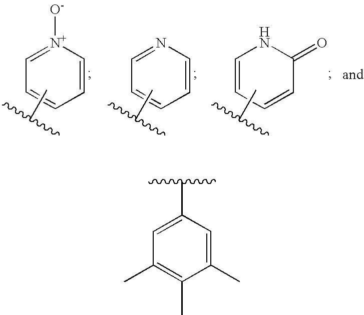
wherein R⁹ and R¹¹ are members independently selected from H, CN, C(O)NR¹²R¹², NR¹²R¹² and OR¹²;
wherein R¹² is a member selected from H, and substituted or unsubstituted C₁-C₄ alkyl;
R¹⁰ is a member selected from CN, NR¹³R¹⁴, SO₂NHR¹³, NHSO₂R¹³, SO₂NH(CH₂)ₙOR¹³, SO₂NH(CH₂)ₙNR¹³R¹⁴, O(CH₂)ₙSO₂NHR¹³, O(CH₂)ₙNR¹³R¹⁴, O(CH₂)ₙSO₂R⁵, SO₂R⁵, SO₂(CH₂)ₙNR¹³R¹⁴ and C(O)NR¹³R¹⁴;
wherein R¹³ and R¹⁴ are members selected from H and substituted or unsubstituted C₁-C₄ alkyl and, together with the nitrogen to which they are attached are optionally joined to form a heterocyclic ring;
R¹⁵ is substituted or unsubstituted C₁-C₄ alkyl;
n is an integer from 1 to 8;
wherein at least one member selected from R⁹ and R¹¹ is other than H; and
R⁷ is a member selected from halogen, C₁-C₄ alkyl, C₂-C₄ alkenyl and C₂-C₄ alkynyl.

2. The compound according to claim 1 wherein R⁶ is a fused phenyl heterocyclic ring system, and the fused phenyl heterocyclic ring system is a member selected from: 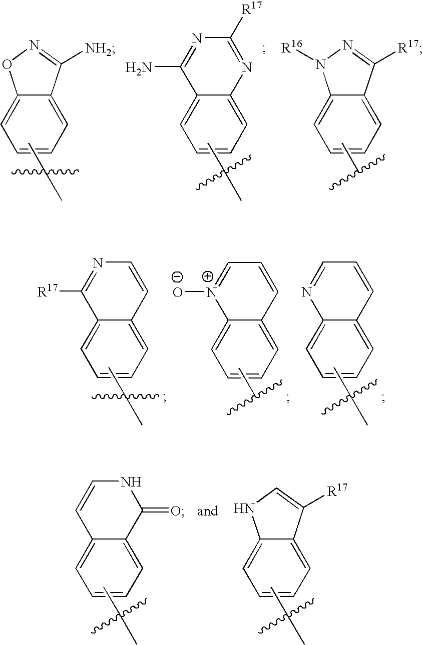
wherein R¹⁶ is a member selected from H, C(O)NR¹⁸R¹⁹, C(O)NR¹⁸ (CH₂)ₙNR¹⁸R¹⁹, C(O)NR¹⁸(CH₂)ₙOR¹⁸, C(O)NR¹⁸CH(CH₂OR¹⁸)₂, C(O)NR¹⁸(CH₂)ₙC(O)NR¹⁸R¹⁹, (CH₂)ₙSO₂NR¹⁸R¹⁹, and S(O)₂R²⁰;
wherein R¹⁸ and R¹⁹ are members independently selected from H and substituted or unsubstituted C₁-C₆ alkyl; or R¹⁸ and R¹⁹, together with the nitrogen atom to which they are both attached, form a substituted or unsubstituted heterocyclic ring;
n is an integer from 1 to 8;
R²⁰ is substituted or unsubstituted C₁-C₆ alkyl and substituted or unsubstituted phenyl; and
R¹⁷ is a member selected from H, NH₂, (CH₂)ₘOH, C(O)NR²¹R²², SO₂R²³, NHSO₂R²³, and NHCOR²³;
wherein R²¹ and R²² are members independently selected from H, substituted or unsubstituted C₁-C₆ alkyl and substituted or unsubstituted phenyl and, together with the nitrogen to which they are attached, are optionally joined to form a ring;
R²³ is substituted or unsubstituted C₁-C₆ alkyl; and
m is an integer from 1 to 5.

3. The compound according to claim 1 in which R¹ and R⁵ are independently selected from hydrogen, halogen, CN, methyl, methoxy, vinyl and trifluoromethoxy.

4. The compound according to claim 1 in which R⁶ is

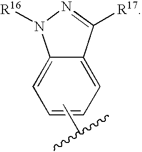

5. The compound according to claim 4 in which R¹⁶ is a member selected from C(O)NR¹⁸R¹⁹ and S(O)₂R²⁰.

6. The compound according to claim 5 wherein R¹⁸ and R¹⁹ are H.

7. The compound according to claim 5 wherein R²⁰ is CH₃.

8. The compound according to claim 4 in which R¹⁷ is NH₂.

9. The compound according to claim 1 wherein R⁶ is 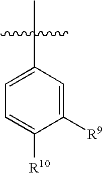
wherein R⁹ is a member selected from NH₂ and C(O)NH₂; and
R¹⁰ is a member selected from C(O)NH₂, NHSO₂CH₃ and SO₂NH₂.

10. The compound according to claim 1 having a formula selected from:

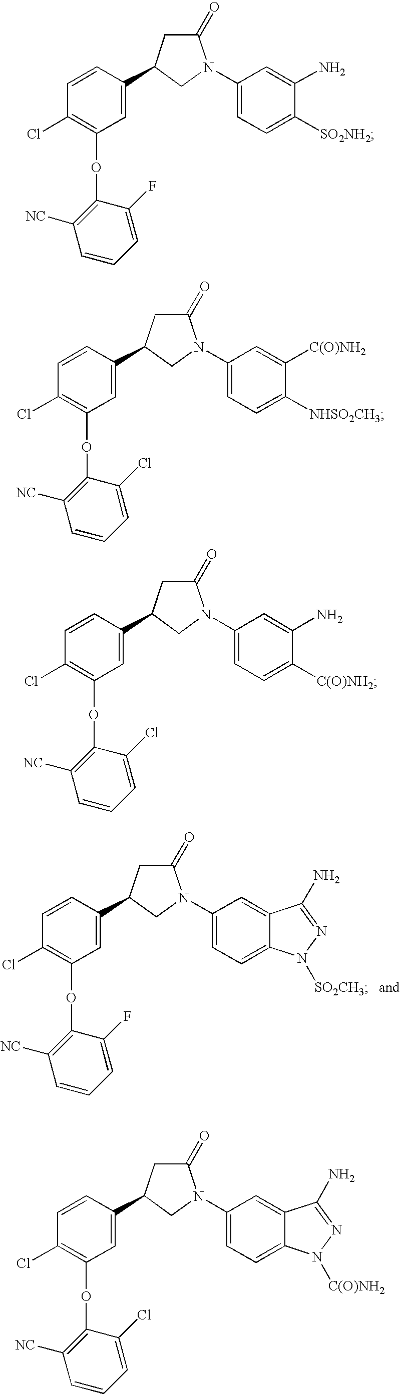

11. A pharmaceutical formulation comprising a compound according to claim 1 and a pharmaceutically acceptable carrier.

12. A method of inhibiting HIV in a cell, said method comprising contacting said cell with an amount of a compound according to claim 1 sufficient to inhibit said HIV.

13. The method according to claim 12, wherein said HIV is a drug resistant strain of HIV.

14. The method according to claim 12, wherein said cell is in a human.

15. A method of inhibiting reverse transcriptase in a cell, said method comprising contacting said cell with an amount of a compound according to claim 1 sufficient to inhibit said reverse transcriptase.

16. The method according to claim 15, wherein said reverse transcriptase is HIV reverse transcriptase.

17. The method according to claim 16 wherein said HIV is a drug resistant strain of HIV.

18. The method according to claim 15, wherein said cell is in a human.

19. A method of treating HIV infection in a human subject comprising administering to said subject an amount of a compound according to claim 1, sufficient to treat said HIV infection.

20. The method according to claim 19, wherein said HIV infection is caused by a drug resistant HIV strain.

21-22. (canceled)

23. The compound according to claim 1, wherein said compound is selected from the group consisting of

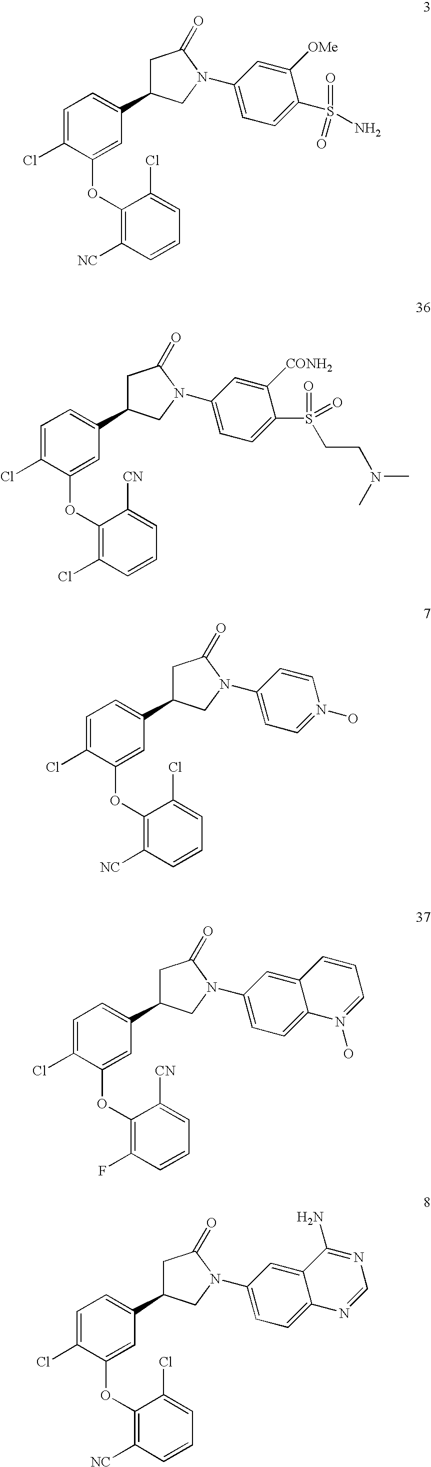

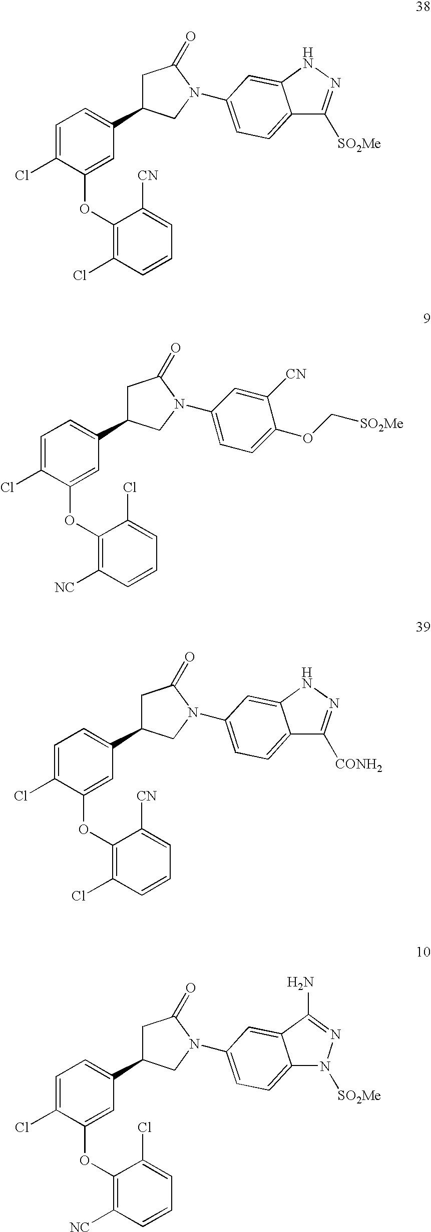

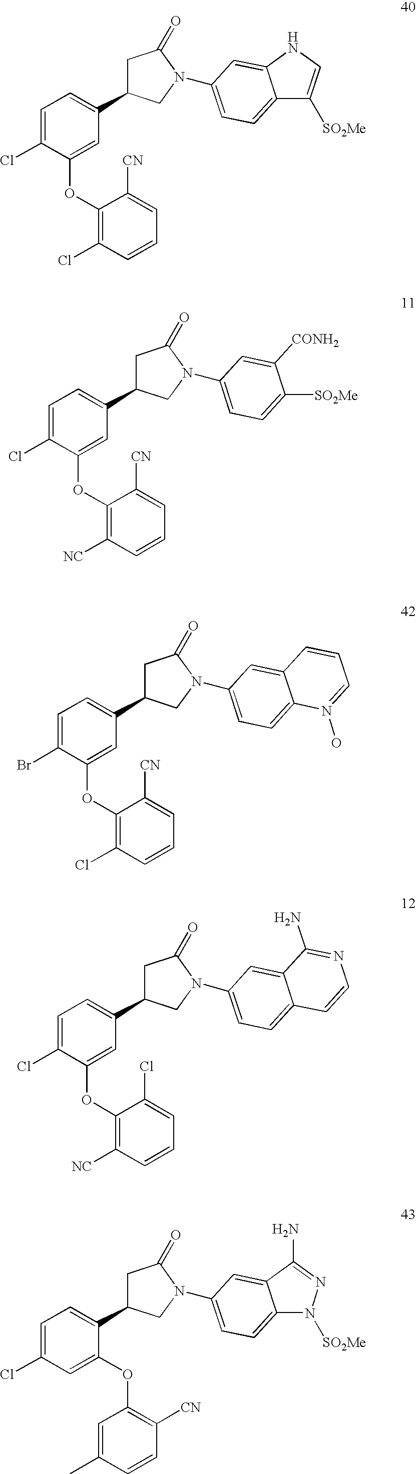

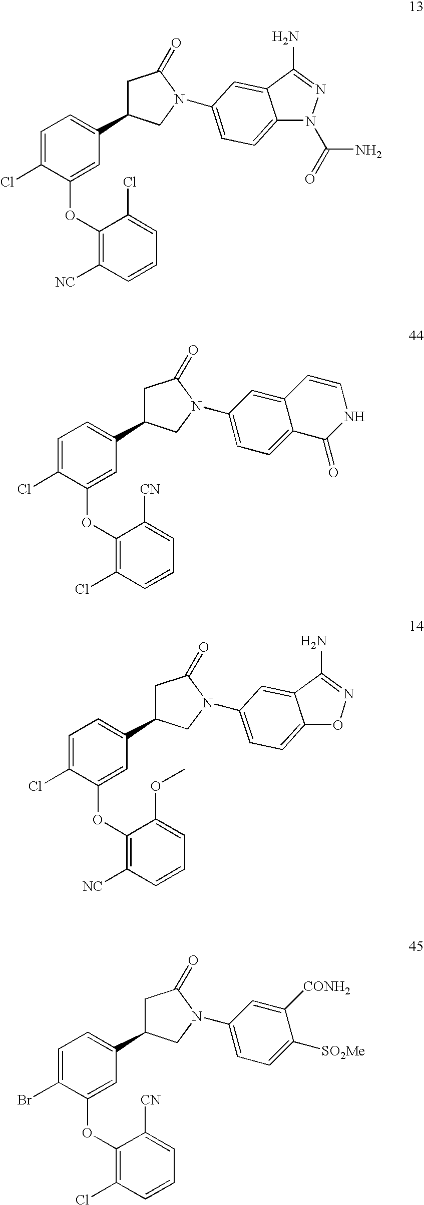

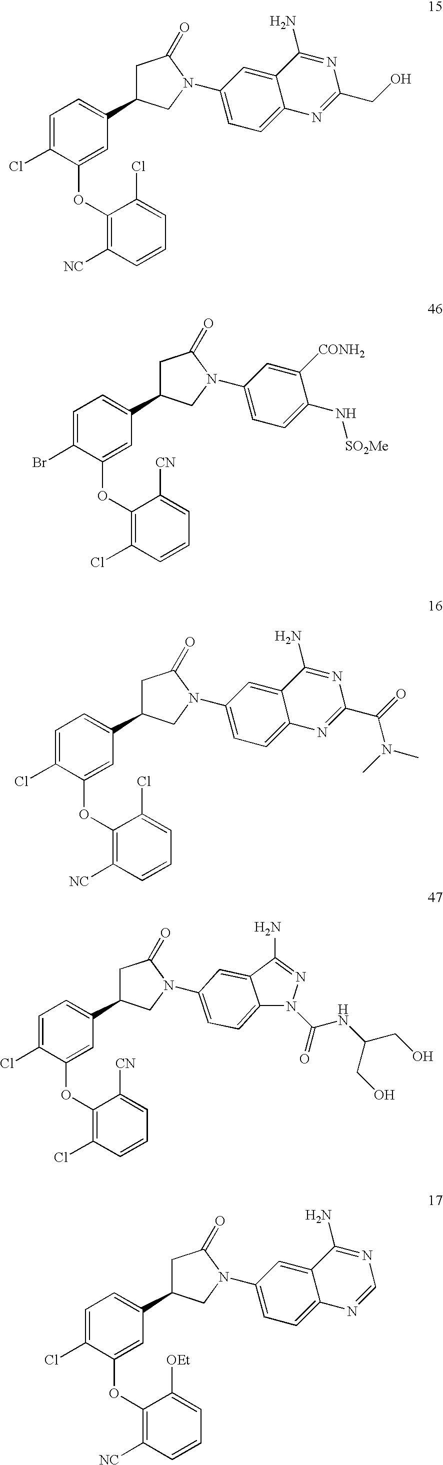

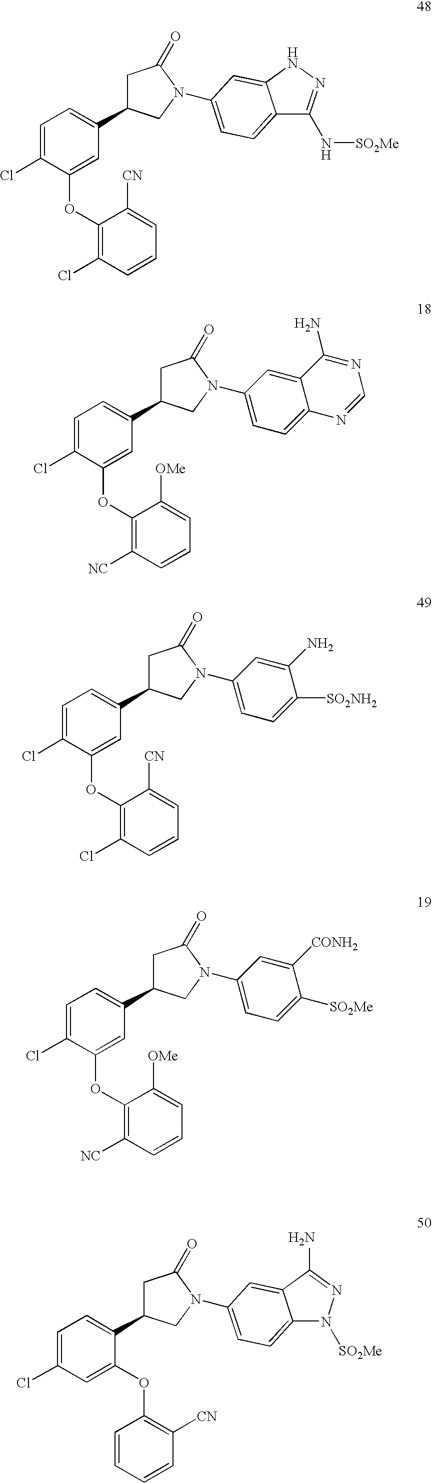

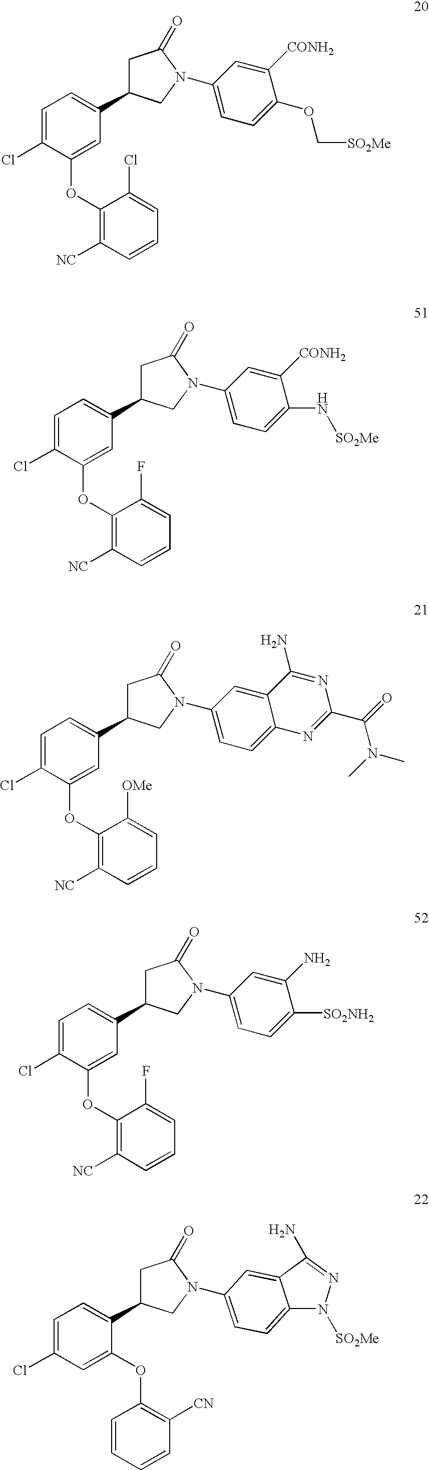

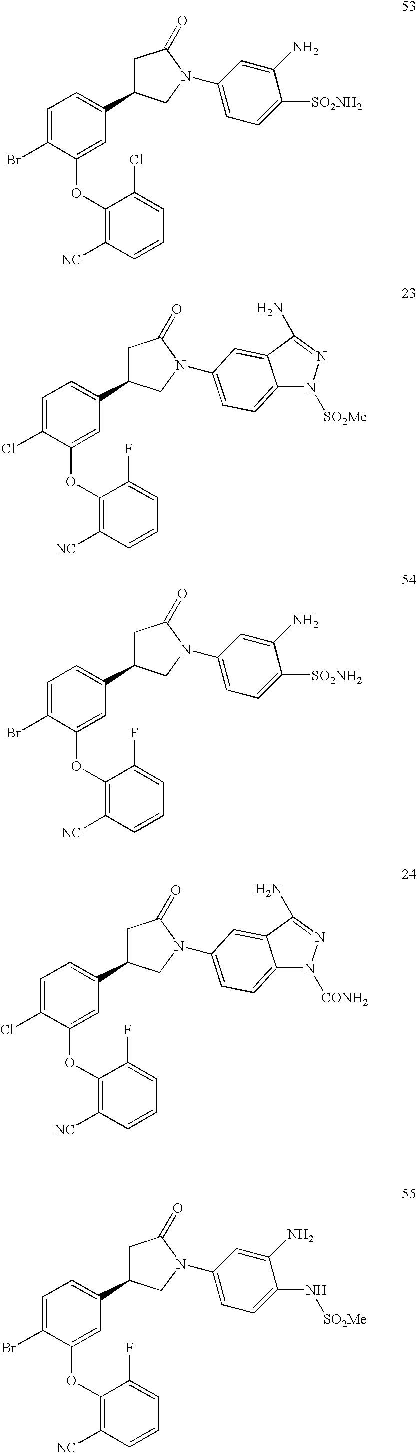

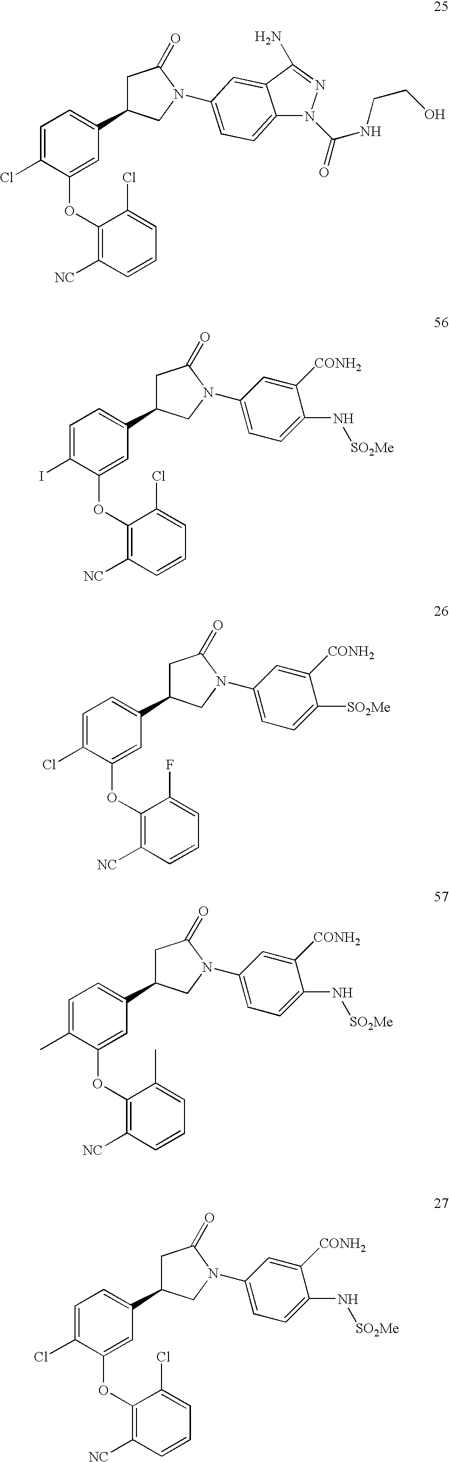

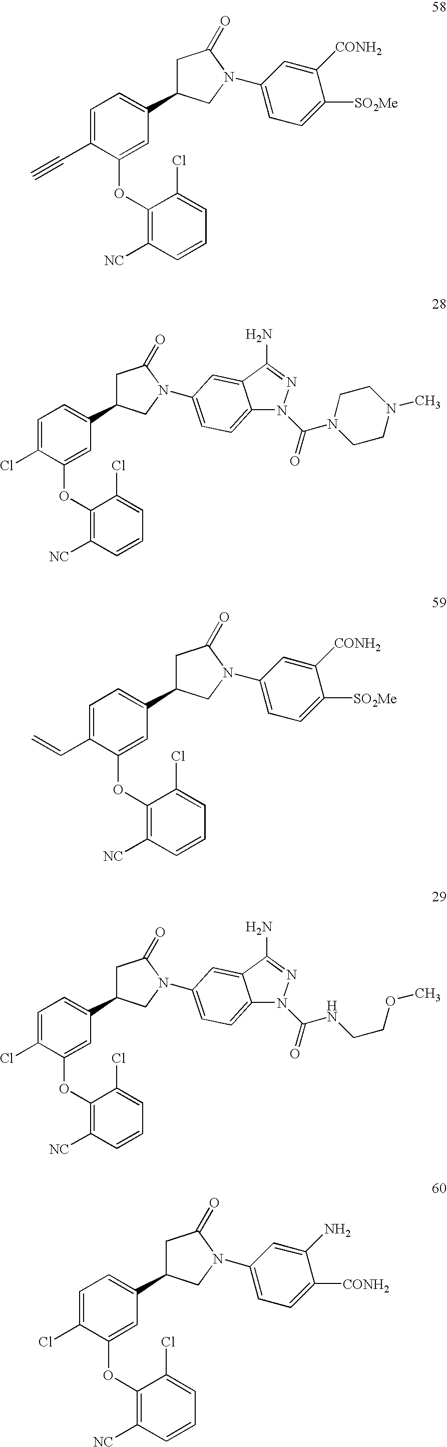

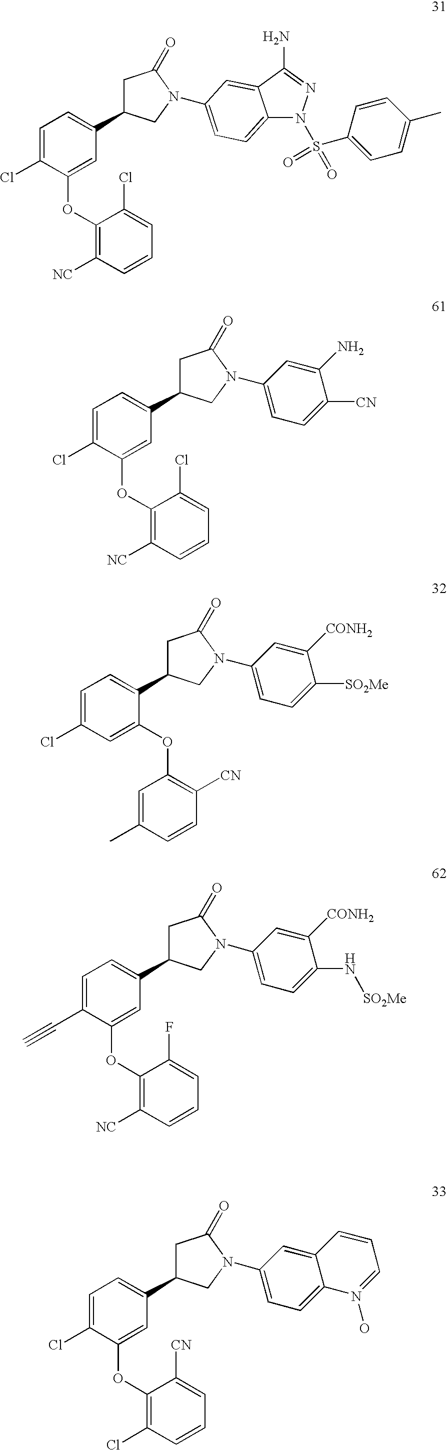

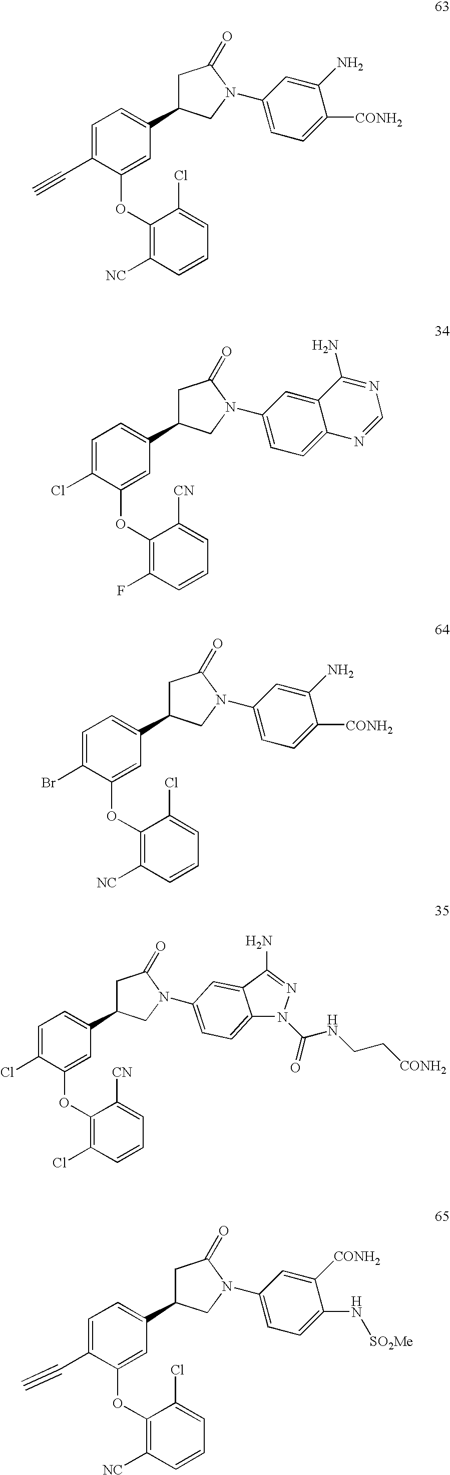

24. A pharmaceutical formulation comprising a compound according to claim 23 and a pharmaceutically acceptable carrier.
